(19) United States
(12) Patent Application Publication
(10) Pub. No.: US 2026/0153487 A1
(43) Pub. Date: Jun. 4, 2026

(54) LIQUID CHROMATOGRAPHY-BASED PROCESS FOR THE CHARACTERIZATION AND QUANTIFICATION OF DOUBLE-STRANDED NUCLEIC ACID

(71) Applicant: Werthenstein BioPharma GmbH, Schachen (CH)

(72) Inventors: Henning Joke Kleine Balderhaar, Menznau (CH); Albert Hutter, Schätz (CH); Jasmin Küng, Goldau (CH)

(73) Assignee: Werthenstein BioPharma GmbH, Schachen (CH)

(21) Appl. No.: 19/400,566

(22) Filed: Nov. 25, 2025

Related U.S. Application Data
(60) Provisional application No. 63/727,382, filed on Dec. 3, 2024.

(51) Int. Cl. G01N 30/88; B01D 15/32; B01D 15/42; C12Q 1/6806; C12Q 1/6869; G01N 30/02

(52) U.S. Cl. CPC G01N 30/88; B01D 15/327; B01D 15/426; C12Q 1/6806; C12Q 1/6869; G01N 2030/027; G01N 2030/8827

(57) ABSTRACT

The present disclosure is directed to a method of quantifying and characterizing double-stranded nucleic acids from a mixture comprising one or more additional nucleic acids or impurities. In some aspects, the method of the present disclosure comprises the steps of separating double-stranded nucleic acids from the mixture, digesting the double-stranded nucleic acids into nucleosides, separating nucleosides by performing at least one step of liquid chromatography, and determining concentration of double-stranded nucleic acids in the mixture. In some embodiments, the liquid chromatography is reverse phase liquid chromatography (RP-LC). In some embodiments, the method further comprises performing at least one step of ion pair reverse phase liquid chromatography (IP-RP-LC) after separating double-stranded nucleic acids from the mixture and before digesting the double-stranded nucleic acids into nucleosides.

### CROSS-REFERENCE TO RELATED APPLICATIONS

This application claims the benefit of U.S. Provisional Patent Application Ser. No. 63/727,382 filed Dec. 3, 2024, the entire contents of which are incorporated by reference herein.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jan. 14, 2025, is named 26086-WO-PCT_SL.xml and is 10,716 bytes in size.

### FIELD

This disclosure relates generally to nucleic acids and analytical methods for characterizing and quantifying the same. More specifically, this disclosure relates to liquid chromatography processes for the characterization and quantification of double-stranded nucleic acid, including ribonucleic acid and deoxyribonucleic acid.

### BACKGROUND

Double-stranded nucleic acid, including double-stranded RNA (dsRNA), is an unwanted by-product of in vitro transcription processes utilized in the production of mRNA therapeutics. Given its prevalence in most viral infections, dsRNA exhibits high immunogenicity, and its cellular detection is a highly conserved feature of the innate immune system. It induces antiviral signaling pathways and inflammatory responses, inhibits cell growth, and can lead to cell death. Hence, monitoring, and quantifying dsRNA has become a regulatory requirement to mitigate the risk of this contaminant and prevent undesirable immunogenic events.

To ensure the highest quality of mRNA products, refined and sophisticated analytical methods have become imperative at all stages of the manufacturing process and final quality control. Currently, dsRNA detection is mostly based on antibody-mediated immunoassays, the performance of which may vary greatly depending upon several factors, including the size range of dsRNA fragments present in a sample, and whether antibody reagents are capable of binding, and thus identifying, each dsRNA fragment size or species within the sample. Thus, there is a need in the art for consistent and reliable assays for monitoring dsRNA contamination of in vitro transcribed therapeutic mRNA products.

The present disclosure provides a liquid chromatography (LC) based methodology for the quality control of in vitro transcribed mRNA. It enables characterization of different double-stranded nucleic acid populations based on their size distribution and allows for quantification of contamination levels through nucleic acid content analysis. The present disclosure shows that the LC assay exhibits high sensitivity and robustness, and is independent of sequence, composition, and size. The present invention thus presents an innovative, effective, and reliable tool for detecting and analyzing double-stranded nucleic acid contaminations in mRNA products.

### SUMMARY

The present disclosure provides a method for quantifying double-stranded nucleic acids from a mixture comprising one or more additional nucleic acids or impurities. In some aspects, the method of the present disclosure comprises the steps of: (a) adding a first enzyme solution to the mixture, wherein the first enzyme solution digests the one or more additional nucleic acids or impurities; (b) separating the double-stranded nucleic acids from the digested one or more additional nucleic acids or impurities; (c) adding a second enzyme solution to the separated double-stranded nucleic acids, wherein the second enzyme solution digests the double-stranded nucleic acids into nucleosides; and (d) analyzing the nucleosides by an LC process to quantify the double-stranded nucleic acids in the mixture.

In some embodiments, the mixture comprising one or more additional nucleic acids or impurities is prepared using an in vitro transcription (IVT) reaction. In some embodiments, the IVT reaction is performed using a T7 polymerase enzyme. In some embodiments, the IVT reaction is performed using one or more oligonucleotide primer selected from the group consisting of SEQ NOs: 1-11.

In some embodiments, the additional nucleic acids in the mixture comprise single-stranded nucleic acids. In some embodiments, the single-stranded nucleic acids are linear or circular. In some embodiments, the single-stranded nucleic acid is mRNA.

In some embodiments, the mRNA comprises a modified nucleotide. In some embodiments, the modified nucleotide is 1-methyl-pseudouridine.

In some embodiments, the first enzyme solution comprises a recombinant protein fusion of RNase I and maltose-binding protein (RNase I_{f}). In some embodiments, the mixture is incubated with the first enzyme solution for about 50 minutes to about 300 minutes. In some embodiments, the mixture is incubated with the first enzyme solution for about 210 minutes.

In some embodiments, the maximal enzyme to substrate ratio is from about 5 units to about 35 units per microgram of substrate. In some embodiments, the maximal enzyme to substrate ratio is 20 units per microgram of substrate.

In some embodiments, following step b, the separated double-stranded nucleic acids comprise less than about 15% of the one or more additional nucleic acids or impurities.

In some embodiments, the second enzyme solution is added to the separated double-stranded nucleic acids for about 30 minutes to about 240 minutes.

In some embodiments, the second enzyme solution digests at least 90% of the double-stranded nucleic acids into nucleosides.

In some embodiments, the LC process is reverse phase liquid chromatography (RP-LC). In some embodiments, the RP-LC process comprises eluting the nucleosides from an RP-LC apparatus with a mobile phase solution comprising a mixture of a first solvent solution and a second solvent solution.

In some embodiments, the first solvent solution comprises one or more solvents selected from the group consisting of: acetonitrile; ammonium acetate; ethanol; formic acid; hexylene glycol; isopropanol; methanol; tetrahydrofuran; and water. In some embodiments, the first solvent solution comprises ammonium acetate and formic acid. In some embodiments, the first solvent solution comprises 20 mM ammonium acetate and 0.1% formic acid.

In some embodiments, the second solvent solution comprises one or more solvents selected from the group consisting of: acetonitrile; ammonium acetate; ethanol; formic acid; hexylene glycol; isopropanol; methanol; tetrahydrofuran; and water. In some embodiments, the second solvent solution comprises methanol.

In some embodiments, the second solvent solution in the mobile phase solution mixture increases in concentration from about 1% to about 100% during the RP-LC process. In some embodiments, the concentration of the second solvent solution in the mobile phase solution mixture: a) increases from about 2% to about 21% over about 6.5 minutes; b) increases from about 21% to about 100% over about 0.45 minutes, and c) decreases from about 100% to about 0% immediately thereafter.

In some embodiments, step d further comprises comparing the double-stranded nucleic acids analyzed by the RP-LC process with reference nucleosides to quantify the double-stranded nucleic acids.

In other aspects of the provided method, step b further comprises an additional step of characterizing a sample of the separated double-stranded nucleic acids by an ion pair reverse phase liquid chromatography (IP-RP-LC) process.

In some embodiments, the IP-RP-LC process comprises eluting the nucleosides from an IP-RP-LC apparatus with a mobile phase solution comprising a mixture of a first solvent solution and a second solvent solution. In some embodiments, the first solvent solution comprises one or more solvents selected from the group consisting of: acetonitrile; ammonium acetate; ethanol; formic acid; hexylene glycol; isopropanol; methanol; tetrahydrofuran; and water. In some embodiments, the first solvent solution comprises one or more ion pairs selected from the group consisting of: hexylammonium acetate; triethylammonium acetate; tetrabutylammonium phosphate; and dibutylammonium acetate.

In some embodiments, the concentration of each of the one or more ion pairs in the first solvent solution is from about 1 mM to about 100 mM.

In some embodiments, the first solvent solution comprises 25 mM hexylammonium acetate.

In some embodiments, the second solvent solution comprises one or more solvents selected from the group consisting of: acetonitrile; ammonium acetate; ethanol; formic acid; hexylene glycol; isopropanol; methanol; tetrahydrofuran; and water.

In some embodiments, the second solvent solution comprises acetonitrile.

In some embodiments, the second solvent solution in the mobile phase solution mixture increases in concentration from about 1% to about 100% during the IP-RP-LC process. In some embodiments, the concentration of the second solvent solution in the mobile phase solution mixture: a) remains at about 5% over about 2.5 minutes; b) increases from about 5% to about 50% over about 12.5 minutes; c) increases from about 40% to about 45% over about 5 minutes; d) increases from about 45% to about 80% over about 5 minutes; e) remains at about 80% for about 5 minutes, and f) decreases from about 80% to about 5% immediately thereafter.

In some embodiments, the double-stranded nucleic acid is linear or circular.

In some embodiments, the double-stranded nucleic acid is dsRNA.

In some embodiments, the double-stranded nucleic acid is dsDNA.

In some embodiments, the double-stranded nucleic acids are about 20 bp to about 5000 bp, as characterized by IP-RP-LC analysis.

In some embodiments, the provided method further comprises one or more steps of determining the nucleic acid sequences of the separated double-stranded nucleic acids.

The summary of the technology described above is non-limiting and other features and advantages of the technology will be apparent from the following detailed description, and from the claims.

### BRIEF DESCRIPTION OF THE DRAWINGS

FIG. 1 is a schematic depiction of the process applied herein to characterize and quantify dsRNA populations by two chromatography methods. Samples containing mRNA were digested with RNase I_{f} (Step 1), followed by a purification and concentration step (Step 2). Isolated dsRNA populations were characterized via IP-RP-LC (Step 3) and quantified via RP-LC (Step 5) after an additional enzyme digestion step (Step 4).

FIGS. 2A and 2B are chromatograms showing the separation of double-stranded polynucleotides and mRNA by IP-RP-LC. FIG. 2A shows an overlay of chromatograms of dsRNA constructs and circular dsDNA plasmid (bottom gray trace), a commercial dsRNA size standard (top gray trace), and a blank separated by IP-RP-LC. FIG. 2B shows an overlay of three commercial mRNA constructs subjected to IP-RP-LC. Absorbance signals were recorded at 260 nm wavelength.

FIG. 3 is a chromatogram showing an overlay of (deoxy) nucleoside mixes (light gray trace=nucleosides; black trace=deoxynucleosides). The inlay illustrates that, depending on the sample, both Uridine and N1-Methyl-Pseudouridine (m1Ψ) could be quantified. Absorbance signals were recorded at 260 nm wavelength.

FIG. 4 is a bar graph showing recovery tests of various dsRNA constructs. Recovery of various dsRNA constructs ranging from 23 bp to 4161 bp, as well as a dsDNA construct of 2686 bp, were tested. The leftmost bars represent the recovery after RNase I_{f} digestion and a subsequent purification step. The rightmost bars indicate the total recovery after RNase I_{f} digestion, purification, and digestion with a nucleoside digestion mix (NDM; New England Biolabs, Ipswich, MA). The middle bars show the isolated efficiency of the NDM step after correction for the determined loss in the first step. Error bars represent the calculated standard deviation including error propagation.

FIGS. 5A-5G are chromatograms showing the integrity of the polynucleotide constructs by IP-RP-LC. Purchased and generated dsRNA constructs (FIGS. 5A-5F), as well as plasmid DNA (FIG. 5G), were tested for integrity before (black traces) and after (light gray traces) incubation with RNase I_{f} and a subsequent purification step. Absorbance signals were recorded at 260 nm wavelength. Asterisks (*) denote the presence of artifact signals from non-analytes at a retention time of approximately 10.5 min.

FIGS. 6A-6G are chromatograms showing the visualization of isolated dsRNA and mRNA populations from mRNA samples analyzed by IP-RP-LC. FIG. 6A shows an overlay of chromatograms of a dsRNA population remaining after RNase I_{f} digestion (light gray trace) and after RNase I_{f}, digestion, purification, and total digestion with an NDM enzyme cocktail to nucleosides (black traces) of mRNA encoding for Cas9. FIG. 6B shows an overlay of chromatograms of a dsRNA population remaining after RNase I_{f} digestion (light gray trace) and after RNase I_{f} digestion, purification, and total digestion with an NDM enzyme cocktail to nucleosides (black traces) of mRNA encoding for firefly luciferase (Fluc). FIG. 6C shows an overlay of chromatograms of a dsRNA population remaining after RNase I_{f} digestion (light gray trace) and after RNase I_{f} digestion, purification, and total digestion with an NDM enzyme cocktail to nucleosides (black traces) of mRNA encoding for eGFP. FIG. 6D shows a chromatogram of a dsRNA standard for determination of respective dsRNA sizes in the samples. FIG. 6E shows controls corresponding to the Cas9 mRNA starting material. FIG. 6F shows controls corresponding to the Fluc mRNA starting material. FIG. 6G shows controls corresponding to the eGFP mRNA starting material. Absorbance signals were recorded at 260 nm wavelength. Asterisks (*) denote the presence of artifact signals from non-analytes at a retention time of approximately 10.5 min.

FIGS. 7A-7C are chromatograms showing representative RP-LC signals of nucleosides and deoxynucleosides derived from tested mRNA constructs. Representative chromatograms indicate separated nucleosides and deoxynucleosides derived from the preparation and testing of Cas9 (FIG. 7A), Fluc (FIG. 7B) and eGFP (FIG. 7C) samples. Absorbance signals were recorded at 260 nm wavelength.

FIGS. 8A-8C are chromatograms showing a dsRNA 142 bp standard assessed by IP-RP-LC before and after purification. FIG. 8A shows a chromatogram of a 142 bp standard as purchased (native). FIG. 8B shows a 142 bp standard material after RNase I_{f} incubation and purification. FIG. 8C shows a typical RP-LC chromatogram of a purified standard material shown in FIG. 8B, indicating the four RNA-derived nucleosides after additional incubation with an NDM enzyme mix. All IP-RP-LC as well as RP-LC signals were recorded at 260 nm wavelength.

FIG. 9 is a bar graph showing the response of ELISA signal relative to dsRNA construct size. dsRNA constructs of varied sizes were subjected to an ELISA assay, using a 142 bp standard as a reference (“REF”). The concentration of the constructs was determined by RP-LC assay and normalized to the concentration of the reference standard. Error bars indicate RSD of triplicate determination for each individual construct. Asterisk (*) indicates that the response level was below detection limit.

### DETAILED DESCRIPTION

The present disclosure is directed to a method of quantifying and characterizing double-stranded nucleic acids from a mixture comprising one or more additional nucleic acids or impurities. In an aspect, the method of the present disclosure comprises the steps of separating double-stranded nucleic acids from the mixture, digesting the double-stranded nucleic acids into nucleosides, separating the nucleosides by performing at least one step of liquid chromatography, and quantifying the double-stranded nucleic acids in the mixture. In some embodiments, the liquid chromatography is RP-LC. In some embodiments, the method further comprises performing at least one step of IP-RP-LC after separating double-stranded nucleic acids from the mixture and before digesting the double-stranded nucleic acids into nucleosides.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

As used herein, the term “nucleic acid” means any DNA- or RNA-molecule and is used synonymously with “polynucleotide.” Furthermore, modifications or derivatives of the nucleic acid as defined herein are explicitly included in the general term “nucleic acid.” For example, peptide nucleic acid (PNA) is also included in the term “nucleic acid.”

“RNA” is the usual abbreviation for ribonucleic acid. It is a nucleic acid molecule, i.e., a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e., ribose, of a first and a phosphate moiety of a second, adjacent monomer. The specific order of the monomers, i.e., the order of the bases linked to the sugar/phosphate-backbone, is called the RNA sequence. Usually, RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as “mRNA.” Processing of the premature RNA, e.g., in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) sequence.

As used herein, the term “RNA” further encompasses RNA molecules, such as viral RNA, retroviral RNA and replicon RNA, small interfering RNA (siRNA), antisense RNA, CRISPR RNA, ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), circular RNA (circRNA), and Piwi-interacting RNA (piRNA).

As used herein, the terms “circRNA” or “circular polyribonucleotide” or “circular RNA” or “oRNA” are used interchangeably and refers to a polyribonucleotide that forms a circular structure through covalent bonds. Circular RNA compositions and methods of making and using the same are described in WO2020237227A1.

As used herein, the term “in vitro transcription” relates to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed and introducing it into an appropriate vector for in vitro transcription, for example into plasmid DNA. The DNA template is often linearized with a suitable restriction enzyme before it is transcribed in vitro. The cDNA may be obtained by reverse transcription of mRNA or chemical synthesis. Moreover, the DNA template for in vitro RNA synthesis may also be obtained by gene synthesis. Methods for in vitro transcription are known in the art.

The term “modified nucleotide” as used herein refers to chemical modifications comprising backbone modifications as well as sugar modifications or base modifications. In some embodiments, modified nucleotides include N1-Methyl-Pseudouridine.

The terms “purification,” “purified” or “purifying” are intended to mean that the dsDNA is separated and/or isolated from a mixture. After the purification step, the dsDNA has a higher purity than before the purification step. The degree of purity after the purification step may be more than 70% or 75%, in particular more than 80% or 85%, very particularly more than 90% or 95% and most favorably 99% or more. The degree of purity may, for example, be determined by an analytical LC method as described herein.

“LC” is the common abbreviation of the term “liquid chromatography.” In LC process a liquid solvent containing the sample mixture is passed through a column filled with a solid adsorbent material leading to the interaction of components of the sample with the adsorbent material. Since different components interact differently with the adsorbent material, this leads to the separation of the components as they flow out of the column. The term LC includes “reversed phase LC (RP-LC),” “ion pair reverse phase liquid chromatography (IP-RP-LC),” size exclusion chromatography, gel filtration, affinity chromatography, or hydrophobic interaction chromatography.

RP-LC uses a non-polar stationary phase and a moderately polar mobile phase and therefore works with hydrophobic interactions which result from repulsive forces between a relatively polar solvent, the relatively non-polar analyte, and the non-polar stationary phase (reversed phase principle). The retention time on the column is therefore longer for molecules which are more non-polar in nature, allowing polar molecules to elute more readily. The retention time is increased by the addition of polar solvent to the mobile phase and decreased by the addition of more hydrophobic solvent.

IP-RP-LC is a specific form of reverse phase LC in which an ion with a lipophilic residue and positive charge such as an alkylammonium salt, e.g., triethylammonium acetate, is added to the mobile phase as counterion.

To “elute” a molecule (e.g., a nucleic acid of interest or an impurity) from a chromatography resin is meant to remove the molecule therefrom by altering the solution conditions such that buffer competes with the molecule of interest for the ligand sites on the chromatography resin. A non-limiting example is to elute a molecule from an liquid chromatography resin by altering the ionic strength of the buffer surrounding the stationary phase of the chromatography material such that the buffer competes with the molecule for the charged sites on the material. Nonlimiting types of elution include isocratic elution and gradient elution, as defined below.

The term “isocratic” is defined herein to denote a chromatographic mobile phase whose composition remains essentially constant for any part of or all of the duration of the chromatographic separation process. The term “isocratic elution” is intended to include a process wherein a single solvent concentration is maintained throughout the separation, a process where the solvent concentration is stepped from one constant concentration to one or more constant concentrations in a sequence of steps, or a process with a gradient separation and with one or more portions conducted under constant solvent concentration conditions.

The term “gradient” or “gradient elution” as defined herein, is a chromatographic mobile phase defined by an initial time point having an initial solvent composition and a final time point having a final solvent composition which is different from the initial solvent concentration, and the gradient composition continually changes (e.g., usually increases) from the initial solvent composition to the final solvent composition over the time interval between the initial and final time points. A gradient mobile phase is used to elute fragments from a chromatography column for the purpose of separating the components in a mixture.

In Vitro Transcription (IVT) of mRNA Therapeutics

“In vitro transcription (IVT)” is a method to synthesize RNA (e.g., therapeutic or antigenic mRNA) from a linear DNA template using an RNA polymerase, where the RNA polymerase binds to a promoter sequence and transcribes mRNA through the addition of complimentary nucleotides in the 5′ to 3′ direction. Linear DNA templates encoding for mRNA sequences are prepared from purified plasmid DNA and may contain an RNA polymerase promoter sequence, encoded 5′ UTR, coding sequence, encoded 3′ UTR with a poly(A) tail, and a linearization cut site.

Plasmid DNA sequences are generated using standard cloning methods in a plasmid vector backbone, such as pUC19, pUC57, or pmRNA^{XP}, and plasmid DNA is purified from fermentation of transformed E. coli strains typically used for plasmid production, such as Stable or DH5a, using standard methods (See, e.g., US Patent Application Publication No. 2019/0083602, US Patent Application Publication No. 2020/0392518). mRNA may be produced in an IVT reaction with a co-transcriptional cap analog addition and a DNA template encoded poly(A) tail, or the cap and/or poly(A) tail may be added enzymatically after the IVT reaction. DNA plasmids may contain an adenine-guanine (AG) sequence at the start of the 5′ UTR after a T7 promoter sequence to enable co-transcriptional capping with a Cap 1 AG analog (m7G(5′)ppp(5′)(2′OMeA)pG). DNA plasmids may also contain a specific restriction enzyme cut site after an encoded 3′ poly(A) tail. Plasmids may be linearized in a digestion reaction containing purified plasmid DNA and the appropriate restriction enzyme in a digest mixture that may contain tris hydrochloride, magnesium, potassium, acetate, albumen, and/or other excipients at a solution pH of about 7-8. The reaction is typically incubated at a temperature range of about 34-40° C. for about 30-90 min.

The linear DNA template may be purified by solvent extraction, alcohol precipitation, centrifugation, chromatographic, and/or filtration-based methods to remove the uncut plasmid DNA, restriction enzyme, and digest mixture components. Purified linear DNA templates may be prepared at any suitable concentration (e.g., about 0.5-2 mg/mL) in water or a buffer containing tris hydrochloride, ethylenediaminetetraacetic acid (EDTA), and/or other excipients at a solution pH of about 7-8.

The IVT reaction is performed in a temperature-controlled reaction vessel with an incubation temperature range of about 34-40° C. The linear DNA template may be added to the IVT reaction mixture at a concentration range of about 10-200 μg/mL. Natural nucleoside triphosphates (NTPs) including adenosine triphosphate (ATP), guanosine triphosphate (GTP), cytidine triphosphate (CTP), uridine triphosphate (UTP), or modified NTPs, such as pseudouridine (Ψ), N1-methylpseudouridine (m1Ψ or 1-methyl-pseudouridine), N6-methyladenosine (m6A), or N5-methylcytidine (m5Ψ), may be used in the IVT reaction.

In some embodiments, one or more mRNAs of the provided method comprise a modified nucleotide. In some embodiments, the modified nucleotide is 1-methyl-pseudouridine.

The NTPs may be incorporated in mRNA as a mixture of natural and substituted modified NTPs, such as a mixture of 50% UTP and 50% m1Ψ. In some embodiments, 100% m¹Ψ may be added to fully substitute for UTP. The NTPs may be added to the IVT reaction mixture at a concentration range of about 2-15 mM for ATP, GTP, CTP and 4-7 mM for m1Ψ. A 5′ Cap 1 AG analog, such as CleanCap AG (3′OMe) (m7 (3′OMeG) (5′) ppp (5′) (2′OMeA) pG) or CleanCap AG (m7 (5′) ppp (5′) (2′OMeA) pG) may be added to the IVT reaction mixture at a concentration range of about 1-10 mM. An RNA polymerase, such as T7 RNA polymerase, may be added to the IVT reaction mixture at a concentration range of about 2,000-20,000 U/mL.

A pyrophosphatase (e.g., yeast inorganic pyrophosphatase) and an RNase inhibitor (e.g., murine RNase inhibitor) may be added to the IVT reaction mixture at a concentration range of 1-5 U/mL and 500-2,000 U/mL, respectively. Other components of the IVT reaction mixture may include tris hydrochloride, magnesium, spermidine, dithiothreitol (DTT), sodium, chloride, potassium, acetate, phosphate, polysorbate-20, polysorbate-80, TritonX-100, glycerol and/or other excipients at a final solution pH of about 6-9. The IVT reaction mixture is typically incubated with mixing for 1-6 hr. In some embodiments, therapeutic or immunogenic mRNA may be produced in the IVT reaction with a co-transcriptional 5′ Cap 1 AG analog and a DNA template encoded 3′ poly(A) tail and no additional enzymatic capping or poly(A) tail addition reactions are necessary. The IVT reaction may be terminated by addition of a DNase, such as DNase I, to digest the linear DNA template into small oligonucleotides and stop transcription. DNase may be added to the IVT reaction mixture at a concentration range of about 100-500 U/mL, along with calcium chloride at a concentration range of about 1-5 mM, and incubated at about 34-40° C. for 0.5-3 hr. After DNase digestion, a proteinase, such as Proteinase K, may be added to the IVT reaction mixture to digest enzymes used in the IVT reaction into small peptides. Proteinase may be added to the IVT reaction mixture at a concentration range of about 0.05-0.2 mg/mL, along with sodium dodecyl sulfate (SDS) and additional DTT, and incubated at about 34-40° C. for 0.5-3 hr. In some embodiments, only DNase treatment is performed, and no proteinase treatment is performed. After DNase or DNase followed by proteinase treatments are complete, the IVT reaction mixture may be adjusted to 10-100 mM EDTA to quench enzyme activity.

The resulting IVT reaction mixture contains the target mRNA sample and contaminants such as template DNA and digested oligonucleotides, enzymes including RNA polymerase, pyrophosphatase, RNase inhibitor, DNase, and proteinase, small molecules including free nucleotides, pyrophosphate, magnesium, spermidine, and/or other IVT reaction matrix excipients, and RNA-related contaminants such as RNA fragments, double-stranded RNA, uncapped RNA, or RNA lacking a poly(A) tail.

In some embodiments, the resulting IVT reaction mixture is contaminated with double-stranded DNA. In some embodiments, the resulting IVT reaction mixture is contaminated with double-stranded RNA. In some embodiments, the double-stranded DNA and/or RNA are about 20 bp to about 5000 bp, as characterized by IP-RP-LC analysis.

An initial tangential flow filtration (TFF) step may be performed after DNase and proteinase treatment for buffer exchange and clearance of small impurities. TFF is operated by pumping a feed solution across a membrane at a transmembrane pressure (TMP) of about 1-10 psi, where solution components larger than the molecular weight cut off (MWCO) remain in the retentate, and components smaller than the MWCO may be permeated through the membrane. The membrane MWCO may range from 30-500 kDa in a flat sheet or hollow fiber format and may be composed of a variety of materials including polyethersulfone (PES), polysulfone (PS), or regenerated cellulose (RC). When the IVT reaction mixture is processed across TFF, the large mRNA molecules are retained while digested impurities and other small molecules are cleared in the permeate. Prior to starting TFF, the IVT reaction mixture may be diluted 2-20-fold in water or a buffer containing tris hydrochloride, sodium phosphate, or sodium citrate at a concentration of about 1-100 mM tris hydrochloride, at a pH of about 6-8 and EDTA at a concentration of about 1-10 mM. A TFF membrane loaded with approximately 1-25 g mRNA per m2 of membrane area of diluted IVT reaction mixture operated at a TMP of about 3-7 psi and crossflow shear rate of about 2000-12000 s-1, may be used to buffer exchange the diluted IVT mixture across 5-15 diafiltration volumes (DVs) into water or a buffer containing tris hydrochloride, sodium phosphate, or sodium citrate at a concentration of about 1-100 mM at a pH of about 6-8 and EDTA at a concentration of about 1-10 mM.

The purified mRNA-containing solution in the TFF retentate may be forwarded to hybridization affinity chromatography media using an oligo deoxythymine (oligo dT) ligand conjugated to a stationary phase to selectively bind mRNA through complimentary base pairing of the oligo dT ligand with the mRNA poly(A) tail. IVT reaction contaminants including RNA polymerase, pyrophosphatase, RNase inhibitor, DNase, and proteinase, small molecules including free nucleotides, pyrophosphate, magnesium, spermidine, other IVT matrix excipients, and RNA fragments lacking a poly(A) tail are not expected to bind to the oligo dT ligand. In some embodiments, the DNase treated IVT reaction mixture may be forwarded directly to oligo dT affinity chromatography, omitting the Proteinase K and initial TFF steps. The oligo dT ligand may consist of about 15-30 deoxythymine bases connected to a linker coupling the ligand to a support surface, such as a microporous polymethacrylate monolith, crosslinked poly(styrene-divinylbenzene) bead, or electrospun cellulose nanofibers. The mRNA-containing solution forwarded to the oligo dT hybridization affinity chromatography step may be diluted about 2-50-fold into an oligo dT binding matrix consisting of a salt, such as sodium chloride, potassium chloride, lithium chloride, guanidine hydrochloride, or other similar salts, at a concentration of about 100-1000 mM, a buffer, such as tris hydrochloride, sodium phosphate, or sodium citrate, at a concentration of 5-100 mM at a pH of about 6-8, and EDTA at a concentration of about 1-10 mM. In some embodiments, the DNase treated IVT mixture may be diluted about 12-fold into an oligo dT binding matrix consisting of 400 mM sodium chloride, 10 mM tris hydrochloride, 2 mM EDTA, pH 7.2. The mRNA-containing solution may be pumped through oligo dT chromatography media with an approximate loading of about 1-8 mg/mL-media at a residence time of about 0.1-10 min, and upon binding of mRNA with a poly(A) tail, the column is washed with 2-10 column volumes (CV) of a mobile phase consisting of a salt, such as sodium chloride, potassium chloride, lithium chloride, guanidine hydrochloride, or other similar salts, at a concentration of about 10-200 mM, a buffer, such as tris hydrochloride, sodium phosphate, or sodium citrate, at a concentration of 5-100 mM at a pH of about 6-8, and EDTA at a concentration of about 1-10 mM. In some embodiments, the mRNA containing mixture may be pumped through a microporous polymethacrylate monolith media at loading of 2-3 mg/mL-media and a residence time of 0.5 min, and the column is washed with 5 CVs of a buffer consisting of 50 mM sodium chloride, 10 mM tris hydrochloride, 2 mM EDTA, pH 7.2. The bound mRNA may be eluted from the oligo dT ligand with 2-10 CVs of a low ionic strength mobile phase consisting of water or a buffer, such as tris hydrochloride, sodium phosphate, or sodium citrate, at a concentration of 1-20 mM at a pH of about 6-8. In some embodiments, the bound mRNA may be eluted with 4-5 CVs of 10 mM tris hydrochloride, pH 7.2. The oligo dT chromatography media may be regenerated with sodium hydroxide and re-used for subsequent chromatography purification cycles. In some embodiments, a polymethacrylate monolith may be regenerated with 0.5 M sodium hydroxide and reused through about 2-6 purification cycles of the same DNase treated IVT mixture diluted in oligo dT binding buffer.

The mRNA-containing oligo dT hybridization affinity chromatography elution fraction may be purified by polishing chromatographic methods to remove residual RNA-related impurities, such as RNA fragments, double-stranded mRNA, and uncapped RNA, or residual enzymes such as RNA polymerase. Examples of polishing chromatographic methods may include mixed-mode chromatography, hydrophobic interaction chromatography, anion-exchange chromatography, reversed-phase chromatography, ceramic hydroxyapatite chromatography, size-exclusion chromatography, or cellulose chromatography. In some embodiments, no additional polishing purification methods are performed after oligo dT chromatography.

The mRNA-containing oligo dT hybridization affinity chromatography elution fraction may be buffer exchanged and purified by a final TFF that may be operated similarly to the previously described initial TFF. The final TFF membrane may be operated at a TMP of 1-10 psi using a 30-500 kDa MWCO membrane in a flat sheet or hollow fiber format and may be composed polyethersulfone (PES), polysulfone (PS), or regenerated cellulose (RC). A 50-100 kDa hollow fiber PES membrane loaded with approximately 1-20 g mRNA per m2 of membrane area operated at a TMP of 3-7 psi and crossflow shear rate of about 2000-12000 s-1 may be used to concentrate the oligo dT elution fraction approximately 2-20-fold followed by a buffer exchange across 4-15 DVs into water or a buffer consisting of about 1-10 mM tris hydrochloride, sodium phosphate, or sodium citrate at a pH of about 5-8.

The mRNA-containing retentate from the final TFF is filtered at a flux of about 25-1000 L/m2-hr and a loading of about 10-1000 g/m² through a bioburden reduction filter with a nominal pore size of about 0.2 μm. The filter may be composed of a variety of materials including hydrophilic polyvinylidene fluoride (PVDF), PES, or cellulose acetate and may contain a prefilter with a nominal pore size ranging from about 0.2-1 μm. The concentration of mRNA in the bioburden reduction filter product is calculated from a sample measurement of absorbance at 260 nm using a spectrophotometer. The filtration product may be diluted with the final TFF diafiltration buffer to a target a final concentration of mRNA ranging from about 0.5-5 mg/mL. The purified mRNA may be stored at refrigerated conditions at about 2-8° C. or frozen at about-20° C. or <−60° C.

### Nucleic Acid

The term “nucleic acid,” in its broadest sense, includes any compound and/or substance that comprises a polymer of nucleotides. These polymers are referred to as “polynucleotides.” Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA), or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA, the “T”s would be substituted for “U”s.

The basic components of an mRNA molecule typically include at least one coding region, a 5′ untranslated region (UTR), a 3′ UTR, a 5′ cap, and a poly(A) tail. Polynucleotides of the present disclosure may function as mRNA but can be distinguished from wild-type mRNA in their functional and/or structural design features which serve to overcome existing problems of effective polypeptide expression using nucleic-acid based therapeutics.

In some embodiments, polynucleotides of the present disclosure are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA), and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence.

In addition to a coding region, a mature mRNA comprises a 5′ untranslated region (UTR) and a 3′ UTR, which play important roles in regulating gene expression. A “5′ UTR” refers to a region of an mRNA that is directly upstream (i.e., 5′) from the start codon (i.e., the first codon of an mRNA transcript translated by a ribosome) that does not encode a polypeptide. A “3′ UTR” refers to a region of an mRNA that is directly downstream (i.e., 3′) from the stop codon (i.e., the codon of an mRNA transcript that signals a termination of translation) that does not encode a polypeptide.

The 5′ UTR may further comprise a 5′ cap sequence. 5′ capping of polynucleotides may be completed concomitantly during an in vitro-transcription reaction using the following chemical RNA cap analogs to generate the 5′ guanosine cap structure according to manufacturer protocols: 3′-O-Me-m7G (5′) ppp (5′) G [the ARCA cap]; G (5′) ppp (5′) A; G (5′) ppp (5′) G; m7G (5′) ppp (5′) A; m7G (5′) ppp (5′) G (New England BioLabs, Ipswich, MA). 5′ capping of modified RNA may be completed post-transcriptionally using a Vaccinia Virus Capping Enzyme to generate the “Cap 0” structure: m7G (5′) ppp (5′) G (New England BioLabs, Ipswich, MA). Cap 1 structure may be generated using both Vaccinia Virus Capping Enzyme and a 2′-O methyl-transferase to generate: m7G (5′) ppp (5′) G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

The 3′ UTR may further comprise a poly(A) sequence or a poly(C) sequence, which are typically located at the 3′ end of an mRNA. A poly(A) sequence, also called poly(A) tail or 3′ poly(A) tail, is typically understood to be a sequence of adenosine nucleotides, e.g., of up to about 400 adenosine nucleotides, e.g., from about 10 to about 400, preferably from about 10 to about 200, more preferably from about 20 to about 80, even more preferably from about 40 to about 80, most preferably from about 60 to about 80 adenosine nucleotides. A poly(C) sequence, also called poly(C) tail or 3′ poly(C) tail, is typically understood to be a sequence of cytidine e nucleotides, e.g., of up to about 400 cytidine nucleotides, e.g., from about 10 to about 400, preferably from about 10 to about 200, more preferably from about 20 to about 80, even more preferably from about 40 to about 80, most preferably from about 60 to about 80 cytidine nucleotides.

In the context of the present invention, a poly(A) sequence may be located within an mRNA or any other nucleic acid molecule, such as, e.g., in a vector, for example, in a vector serving as template for the generation of an RNA, preferably an mRNA, e.g., by transcription of the vector. Moreover, poly(A) sequences, or poly(A) tails may be generated in vitro by enzymatic polyadenylation of the RNA, e.g., using Poly(A) polymerases (PAP) derived from E. coli or yeast. In addition, polyadenylation of RNA can be achieved by using immobilized PAP enzymes e.g., in a polyadenylation reactor. See, International Patent Application Publication WO 2016/174271.

### Modified Nucleotides

RNAs of the present disclosure may comprise an open reading frame comprising at least one modified nucleotide. The terms “modified nucleotide,” “nucleic acid modification,” “chemical modification,” and “chemically modified” refer to modification with respect to adenosine (A), guanosine (G), uridine (U), thymidine (T), or cytidine (C) ribonucleosides or deoxyribonucleosides in at least one of their position, pattern, percent, or population. Generally, these terms do not refer to the ribonucleotide modifications in naturally occurring 5′ terminal mRNA cap moieties. With respect to a polypeptide, the term “modification” refers to a modification relative to the canonical set 20 amino acids. Polypeptides, as provided herein, are also considered “modified” if they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two, or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine, or uracil. Any combination of base/sugar or linker may be incorporated into polynucleotides of the present disclosure.

Modifications of polynucleotides (e.g., RNA polynucleotides, such as mRNA polynucleotides) that are useful in the present disclosure include, but are not limited to the following: 2-methylthio-N6-(cis-hydroxyisopentenyl) adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl) adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl) adenine; N6-cis-hydroxy-isopentenyl-adenosine; α-thio-adenosine; 2 (amino) adenine; 2 (aminopropyl) adenine; 2 (methylthio) N6 (isopentenyl) adenine; 2-(alkyl) adenine; 2-(aminoalkyl) adenine; 2-(aminopropyl) adenine; 2-(halo) adenine; 2-(halo) adenine; 2-(propyl) adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl) adenine; 6 (methyl) adenine; 6-(alkyl) adenine; 6-(methyl) adenine; 7 (deaza) adenine; 8 (alkenyl) adenine; 8 (alkynyl) adenine; 8 (amino) adenine; 8 (thioalkyl) adenine; 8-(alkenyl) adenine; 8-(alkyl) adenine; 8-(alkynyl) adenine; 8-(amino) adenine; 8-(halo) adenine; 8-(hydroxyl) adenine; 8-(thioalkyl) adenine; 8-(thiol) adenine; 8-azido-adenosine; aza adenine; deaza adenine; N6 (methyl) adenine; N6-(isopentyl) adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-α-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio) cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza) cytosine; 3 (methyl) cytosine; 3-(alkyl) cytosine; 3-(deaza) 5 (aza) cytosine; 3-(methyl) cytidine; 4,2′-O-dimethylcytidine; 5 (halo) cytosine; 5 (methyl) cytosine; 5 (propynyl) cytosine; 5 (trifluoromethyl) cytosine; 5-(alkyl) cytosine; 5-(alkynyl) cytosine; 5-(halo) cytosine; 5-(propynyl) cytosine; 5-(trifluoromethyl) cytosine; 5-bromo-cytidine; 5-iodo-cytidine; 5-propynyl cytosine; 6-(azo) cytosine; 6-aza-cytidine; aza cytosine; deaza cytosine; N4 (acetyl) cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl) cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-α-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl) cytidine TP; 3′-Ethynylcytidine TP; 4′-Azidocytidine TP; 4′-Carbocyclic cytidine TP; 4′-Ethynylcytidine TP; 5-(1-Propynyl) ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2, N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl) guanine; 2-(alkyl) guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl) guanine; 6-(alkyl) guanine; 6-(methyl) guanine; 6-methyl-guanosine; 7 (alkyl) guanine; 7 (deaza) guanine; 7 (methyl) guanine; 7-(alkyl) guanine; 7-(deaza) guanine; 7-(methyl) guanine; 8 (alkyl) guanine; 8 (alkynyl) guanine; 8 (halo) guanine; 8 (thioalkyl) guanine; 8-(alkenyl) guanine; 8-(alkyl) guanine; 8-(alkynyl) guanine; 8-(amino) guanine; 8-(halo) guanine; 8-(hydroxyl) guanine; 8-(thioalkyl) guanine; 8-(thiol) guanine; aza guanine; deaza guanine; N (methyl) guanine; N-(methyl) guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-α-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl) uridine; 1-methyl-3-(3-amino-5-carboxypropyl) pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl) uridine; 3,2′-O-dimethyluridine; 3-Methyl-pseudo-Uridine TP; 4-thiouridine; 5-(carboxyhydroxymethyl) uridine; 5-(carboxyhydroxymethyl) uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl) uridine TP; 5-propynyl uracil; α-thio-uridine; 1 (aminoalkylamino-carbonylethylenyl)-2 (thio)-pseudouracil; 1 (aminoalkylaminocarbonylethylenyl)-2,4-(dithio) pseudouracil; 1 (aminoalkylaminocarbonylethylenyl)-4 (thio) pseudouracil; 1 (aminoalkylaminocarbonylethylenyl)-pseudouracil; 1 (aminocarbonylethylenyl)-2 (thio)-pseudouracil; 1 (aminocarbonylethylenyl)-2,4-(dithio) pseudouracil; 1 (aminocarbonylethylenyl)-4 (thio) pseudouracil; 1 (aminocarbonylethylenyl)-pseudouracil; 1 substituted 2 (thio)-pseudouracil; 1 substituted 2,4-(dithio) pseudouracil; 1 substituted 4 (thio) pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl) pseudo-UTP; 1-Methyl-pseudo-UTP; 2 (thio) pseudouracil; 2′ deoxy uridine; 2′ fluorouridine; 2-(thio) uracil; 2,4-(dithio) psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl) uracil; 4 (thio) pseudouracil; 4-(thio) pseudouracil; 4-(thio) uracil; 4-thiouracil; 5 (1,3-diazole-1-alkyl) uracil; 5 (2-aminopropyl) uracil; 5 (aminoalkyl) uracil; 5 (dimethylaminoalkyl) uracil; 5 (guanidiniumalkyl) uracil; 5 (methoxycarbonylmethyl)-2-(thio) uracil; 5 (methoxycarbonyl-methyl) uracil; 5 (methyl) 2 (thio) uracil; 5 (methyl) 2,4 (dithio) uracil; 5 (methyl) 4 (thio) uracil; 5 (methylaminomethyl)-2 (thio) uracil; 5 (methylaminomethyl)-2,4 (dithio) uracil; 5 (methylaminomethyl)-4 (thio) uracil; 5 (propynyl) uracil; 5 (trifluoromethyl) uracil; 5-(2-aminopropyl) uracil; 5-(alkyl)-2-(thio) pseudouracil; 5-(alkyl)-2,4 (dithio) pseudouracil; 5-(alkyl)-4 (thio) pseudouracil; 5-(alkyl) pseudouracil; 5-(alkyl) uracil; 5-(alkynyl) uracil; 5-(allylamino) uracil; 5-(cyanoalkyl) uracil; 5-(dialkylaminoalkyl) uracil; 5-(dimethylaminoalkyl) uracil; 5-(guanidiniumalkyl) uracil; 5-(halo) uracil; 5-(1,3-diazole-1-alkyl) uracil; 5-(methoxy) uracil; 5-(methoxycarbonylmethyl)-2-(thio) uracil; 5-(methoxycarbonyl-methyl) uracil; 5-(methyl) 2 (thio) uracil; 5-(methyl) 2,4 (dithio) uracil; 5-(methyl) 4 (thio) uracil; 5-(methyl)-2-(thio) pseudouracil; 5-(methyl)-2,4 (dithio) pseudouracil; 5-(methyl)-4 (thio) pseudouracil; 5-(methyl) pseudouracil; 5-(methylaminomethyl)-2 (thio) uracil; 5-(methylaminomethyl)-2,4 (dithio) uracil; 5-(methylaminomethyl)-4-(thio) uracil; 5-(propynyl) uracil; 5-(trifluoromethyl) uracil; 5-aminoallyl-uridine; 5-bromo-uridine; 5-iodo-uridine; 5-uracil; 6 (azo) uracil; 6-(azo) uracil; 6-aza-uridine; allyamino-uracil; aza uracil; deaza uracil; N3 (methyl) uracil; P seudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+) 1-(2-Hydroxypropyl) pseudouridine TP; (2R)-1-(2-Hydroxypropyl) pseudouridine TP; (2S)-1-(2-Hydroxypropyl) pseudouridine TP; (E)-5-(2-Bromo-vinyl) ara-uridine TP; (E)-5-(2-Bromo-vinyl) uridine TP; (Z)-5-(2-Bromo-vinyl) ara-uridine TP; (Z)-5-(2-Bromo-vinyl) uridine TP; 1-(2,2,2-Trifluoroethyl)-pseudo-UTP; 1-(2,2,3,3,3-Pentafluoropropyl) pseudouridine TP; 1-(2,2-Diethoxyethyl) pseudouridine TP; 1-(2,4,6-Trimethylbenzyl) pseudouridine TP; 1-(2,4,6-Trimethyl-benzyl) pseudo-UTP; 1-(2,4,6-Trimethyl-phenyl) pseudo-UTP; 1-(2-Amino-2-carboxyethyl) pseudo-UTP; 1-(2-Amino-ethyl) pseudo-UTP; 1-(2-Hydroxyethyl) pseudouridine TP; 1-(2-Methoxyethyl) pseudouridine TP; 1-(3,4-Bis-trifluoromethoxybenzyl) pseudouridine TP; 1-(3,4-Dimethoxybenzyl) pseudouridine TP; 1-(3-Amino-3-carboxypropyl) pseudo-UTP; 1-(3-Amino-propyl) pseudo-UTP; 1-(3-Cyclopropyl-prop-2-ynyl) pseudouridine TP; 1-(4-Amino-4-carboxybutyl) pseudo-UTP; 1-(4-Amino-benzyl) pseudo-UTP; 1-(4-Amino-butyl) pseudo-UTP; 1-(4-Amino-phenyl) pseudo-UTP; 1-(4-Azidobenzyl) pseudouridine TP; 1-(4-Bromobenzyl) pseudouridine TP; 1-(4-Chlorobenzyl) pseudouridine TP; 1-(4-Fluorobenzyl) pseudouridine TP; 1-(4-lodobenzyl) pseudouridine TP; 1-(4-Methanesulfonylbenzyl) pseudouridine TP; 1-(4-Methoxybenzyl) pseudouridine TP; 1-(4-Methoxy-benzyl) pseudo-UTP; 1-(4-Methoxy-phenyl) pseudo-UTP; 1-(4-Methylbenzyl) pseudouridine TP; 1-(4-Methyl-benzyl) pseudo-UTP; 1-(4-Nitrobenzyl) pseudouridine TP; 1-(4-Nitro-benzyl) pseudo-UTP; 1 (4-Nitro-phenyl) pseudo-UTP; 1-(4-Thiomethoxybenzyl) pseudouridine TP; 1-(4-Trifluoromethoxybenzyl) pseudouridine TP; 1-(4-Trifluoromethylbenzyl) pseudouridine TP; 1-(5-Amino-pentyl) pseudo-UTP; 1-(6-Amino-hexyl) pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl) pseudo-UTP; 1-Methyl-6-(4-morpholino)-pseudo-UTP; 1-Methyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-α-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl) uridine TP; 3-Alkyl-pseudo-UTP; 4′-Azidouridine TP; 4′-Carbocyclic uridine TP; 4′-Ethynyluridine TP; 5-(1-Propynyl) ara-uridine TP; 5-(2-Furanyl) uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2 (2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino) purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino) purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine;

Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl) uridine TP; and N6-(19-Amino-pentaoxanonadecyl) adenosine TP.

In some embodiments, polynucleotides (e.g., RNA polynucleotides, such as mRNA polynucleotides) include at least one (e.g., 1, 2, 3, 4, or more) of the aforementioned modified nucleobases.

In some embodiments, modified nucleobases in polynucleotides (e.g., RNA polynucleotides, such as mRNA polynucleotides) are selected from the group consisting of pseudouridine (Ψ), N1-methylpseudouridine (m¹Ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include at least one (e.g., 1, 2, 3, 4, or more) of the aforementioned modified nucleobases.

In some embodiments, one or more polynucleotide of the invention is modified by replacing one or more uridine residue with one or more modified nucleobase. In certain embodiments, at least 50% of the uridine residues of the mRNA polynucleotide are replaced with N1-methylpseudouridine. In certain embodiments, between 50% and 55%, between 55% and 60%, between 60% and 65%, between 65% and 70%, between 70% and 75%, between 75% and 80%, 80% and 85%, between 85% and 90%, between 90% and 95%, and between 95% and 100% of the uridine residues of the mRNA polynucleotide are replaced with N1-methylpsuedouridine. In certain embodiments, 90% of the uridine residues of the mRNA polynucleotide are replaced with N1-methylpsuedouridine. In certain embodiments, 91% of the uridine residues of the mRNA polynucleotide are replaced with N1-methylpsuedouridine. In certain embodiments, 92% of the uridine residues of the mRNA polynucleotide are replaced with N1-methylpsuedouridine. In certain embodiments, 93% of the uridine residues of the mRNA polynucleotide are replaced with N1-methylpsuedouridine. In certain embodiments, 94% of the uridine residues of the mRNA polynucleotide are replaced with N1-methylpsuedouridine. In certain embodiments, 95% of the uridine residues of the mRNA polynucleotide are replaced with N1-methylpsuedouridine. In certain embodiments, 96% of the uridine residues of the mRNA polynucleotide are replaced with N1-methylpsuedouridine. In certain embodiments, 97% of the uridine residues of the mRNA polynucleotide are replaced with N1-methylpsuedouridine. In certain embodiments, 98% of the uridine residues of the mRNA polynucleotide are replaced with N1-methylpsuedouridine. In certain embodiments, 99% of the uridine residues of the mRNA polynucleotide are replaced with N1-methylpsuedouridine In certain embodiments, 100% of the uridine residues of the mRNA polynucleotide are replaced with N1-methylpsuedouridine.

In some embodiments, modified nucleobases in polynucleotides (e.g., RNA polynucleotides, such as mRNA polynucleotides) are selected from the group consisting of 1-methyl-pseudouridine (m¹Ψ), 5-methoxy-uridine (mo⁵U), 5-methyl-cytidine (m5Ψ), pseudouridine (Ψ), α-thio-guanosine and α-thio-adenosine. In some embodiments, polynucleotides include at least one (e.g., 1, 2, 3, 4, or more) of the aforementioned modified nucleobases.

In some embodiments, polynucleotides (e.g., RNA polynucleotides, such as mRNA polynucleotides) comprise pseudouridine (Ψ) and 5-methyl-cytidine (m5C). In some embodiments, polynucleotides (e.g., RNA polynucleotides, such as mRNA polynucleotides) comprise 1-methyl-pseudouridine (m1Ψ). In some embodiments, polynucleotides (e.g., RNA polynucleotides, such as mRNA polynucleotides) comprise 1-methyl-pseudouridine (m1Ψ) and 5-methyl-cytidine (m5C). In some embodiments, polynucleotides (e.g., RNA polynucleotides, such as mRNA polynucleotides) comprise 2-thiouridine (s2U). In some embodiments, polynucleotides (e.g., RNA polynucleotides, such as mRNA polynucleotides) comprise 2-thiouridine and 5-methyl-cytidine (m5C). In some embodiments, polynucleotides (e.g., RNA polynucleotides, such as mRNA polynucleotides) comprise methoxy-uridine (mo5U). In some embodiments, polynucleotides (e.g., RNA polynucleotides, such as mRNA polynucleotides) comprise 5-methoxy-uridine (mo5U) and 5-methyl-cytidine (m5C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m5C). In some embodiments, polynucleotides (e.g., RNA polynucleotides, such as mRNA polynucleotides) comprise N6-methyl-adenosine (m6A). In some embodiments, polynucleotides (e.g., RNA polynucleotides, such as mRNA polynucleotides) comprise N6-methyl-adenosine (m6A) and 5-methyl-cytidine (m5C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m5Ψ), meaning that all cytosine residues in the mRNA sequence are replaced with 5-methyl-cytidine (m5Ψ). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified cytosine, or, a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the poly(A) tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 1, 2, 3, 4, or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 1, 2, 3, 4, or more unique structures).

### Liquid Chromatography (LC)

The present disclosure is directed to a method of quantifying and characterizing double-stranded nucleic acids from a mixture comprising one or more additional nucleic acids or impurities. In an aspect of the present disclosure, the method comprises, inter alia, a step of separating double-stranded nucleic acids from a mixture comprising one or more additional nucleic acids or impurities. The method of the present disclosure comprises the steps of: (a) adding a first enzyme solution to the mixture, wherein the first enzyme solution digests the one or more additional nucleic acids or impurities; (b) separating the double-stranded nucleic acids from the digested one or more additional nucleic acids or impurities; (c) adding a second enzyme solution to the separated double-stranded nucleic acids, wherein the second enzyme solution digests the double-stranded nucleic acids into nucleosides; and (d) analyzing the nucleosides by a liquid chromatography process to quantify the double-stranded nucleic acids in the mixture.

In some embodiments, the first enzyme solution comprises a recombinant fusion protein comprising RNase I and maltose-binding protein domain (RNase I_{f}).

In some embodiments, the mixture is incubated with the first enzyme solution for about 10 minutes to about 500 minutes, e.g., about 10 to about 20 minutes, about 20 to about 30 minutes, about 30 to about 40 minutes, about 40 to about 50 minutes, about 50 to about 60 minutes, about 60 to about 70 minutes, about 70 to about 80 minutes, about 80 to about 90 minutes, about 90 to about 100 minutes; about 100 to about 110 minutes, 110 to about 120 minutes, about 120 to about 130 minutes, about 130 to about 140 minutes, about 140 to about 150 minutes, about 150 to about 160 minutes, about 160 to about 170 minutes, about 170 to about 180 minutes, about 180 to about 190 minutes, about 190 to about 200 minutes; about 200 to about 210 minutes, 210 to about 220 minutes, about 220 to about 230 minutes, about 230 to about 240 minutes, about 240 to about 250 minutes, about 250 to about 260 minutes, about 260 to about 270 minutes, about 270 to about 280 minutes, about 280 to about 290 minutes, about 290 to about 300 minutes; about 300 to about 310 minutes, 310 to about 320 minutes, about 320 to about 330 minutes, about 330 to about 340 minutes, about 340 to about 350 minutes, about 350 to about 360 minutes, about 360 to about 370 minutes, about 370 to about 380 minutes, about 380 to about 390 minutes, about 390 to about 400 minutes; about 400 to about 410 minutes, 410 to about 420 minutes, about 420 to about 430 minutes, about 430 to about 440 minutes, about 440 to about 450 minutes, about 450 to about 460 minutes, about 460 to about 470 minutes, about 470 to about 480 minutes, about 480 to about 490 minutes, or about 490 to about 500 minutes.

In some embodiments, the mixture is incubated with the first enzyme solution for about 50 minutes to about 300 minutes. In some embodiments, the mixture is incubated with the first enzyme solution for about 210 minutes.

In some embodiments, the maximal enzyme to substrate ratio is from about 1 unit to about 100 units per microgram of substrate, e.g., about 1 unit to about 10 units, about 10 units to about 20 units, about 20 units to about 30 units, about 30 units to about 40 units, about 40 units to about 50 units, about 50 units to about 60 units, about 60 units to about 70 units, about 70 units to about 80 units, about 80 units to about 90 units, or about 90 units to about 100 units per microgram of substrate.

In some embodiments, the maximal enzyme to substrate ratio is from about 5 units to about 35 units per microgram of substrate, e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 units per microgram of substrate. In some embodiments, the maximal enzyme to substrate ratio is 20 units per microgram of substrate.

In some embodiments, the separated double-stranded nucleic acids comprise less than about 25% of the one or more additional nucleic acids or impurities. In some embodiments, the separated double-stranded nucleic acids comprise less than about 15% of the one or more additional nucleic acids or impurities. In some embodiments, the separated double-stranded nucleic acids comprise less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, or less than 0.1% of the one or more additional nucleic acids or impurities.

In some embodiments, a second enzyme solution is added to the separated double-stranded nucleic acids. Any member of any class of enzyme or reagent which is capable of generating single nucleosides from DNA or RNA may be suitable for use in the provided method. In some embodiments, the second enzyme solution comprises a commercial nucleoside digestion mix.

In some embodiments, the second enzyme solution is added to the separated double-stranded nucleic acids for about 10 minutes to about 500 minutes, e.g., about 10 to about 20 minutes, about 20 to about 30 minutes, about 30 to about 40 minutes, about 40 to about 50 minutes, about 50 to about 60 minutes, about 60 to about 70 minutes, about 70 to about 80 minutes, about 80 to about 90 minutes, about 90 to about 100 minutes; about 100 to about 110 minutes, 110 to about 120 minutes, about 120 to about 130 minutes, about 130 to about 140 minutes, about 140 to about 150 minutes, about 150 to about 160 minutes, about 160 to about 170 minutes, about 170 to about 180 minutes, about 180 to about 190 minutes, about 190 to about 200 minutes; about 200 to about 210 minutes, 210 to about 220 minutes, about 220 to about 230 minutes, about 230 to about 240 minutes, about 240 to about 250 minutes, about 250 to about 260 minutes, about 260 to about 270 minutes, about 270 to about 280 minutes, about 280 to about 290 minutes, about 290 to about 300 minutes; about 300 to about 310 minutes, 310 to about 320 minutes, about 320 to about 330 minutes, about 330 to about 340 minutes, about 340 to about 350 minutes, about 350 to about 360 minutes, about 360 to about 370 minutes, about 370 to about 380 minutes, about 380 to about 390 minutes, about 390 to about 400 minutes; about 400 to about 410 minutes, 410 to about 420 minutes, about 420 to about 430 minutes, about 430 to about 440 minutes, about 440 to about 450 minutes, about 450 to about 460 minutes, about 460 to about 470 minutes, about 470 to about 480 minutes, about 480 to about 490 minutes, or about 490 to about 500 minutes.

In some embodiments, the second enzyme solution is added to the separated double-stranded nucleic acids for about 30 minutes to about 240 minutes.

In some embodiments, the second enzyme solution digests at least 70% of the double-stranded nucleic acids into nucleosides. In some embodiments, the second enzyme solution digests at least 80% of the double-stranded nucleic acids into nucleosides. In some embodiments, the second enzyme solution digests at least 90%, e.g., at least 90%, 91%, 92%, 93%, 94%, 95%, 96%, 97%, 98%, or 99% of the double-stranded nucleic acids into nucleosides.

Any suitable method for separating RNA fragments can be used, including but not limited to, denaturing gel electrophoresis or liquid chromatography. Separation of RNA molecules by denaturing gel electrophoresis has been described. See, e.g., Maniatis et al., 1975. Biochemistry 14 (17): 3787-3794). For example, polyacrylamide gels that contain a high concentration of a denaturing agent such as urea are capable of resolving short (<500 nucleotides) single-stranded RNA fragments that differ in length by as little as one nucleotide.

RNA molecules may also be separated by liquid chromatography methods. As used herein, the term “liquid chromatography (LC)” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., the mobile phase), as this fluid moves relative to the “stationary phases.” Stationary phases for the use in liquid chromatography are known in the art. For example, the stationary phase can be a porous polystyrene, a porous non-alkylated polystyrene, a polystyrenedi-vinylbenzene, a porous non-alkylated polystyrenedivinylbenzene, a porous silica gel, a porous silica gel modified with non-polar residues, a porous silica gel modified with alkyl containing residues, selected from butyl-, octyl and/or octadecyl containing residues, a porous silica gel modified with phenylic residues, or a porous polymethacrylate (See, WO2008077592).

LC includes high performance liquid chromatography (HP-LC), high turbulence liquid chromatography (HT-LC), fast performance liquid chromatography (FPLC), reverse phase liquid chromatography (RP-LC), and ion pair reverse phase liquid chromatography (IP-RP-LC). The separation of RNA molecules by RP-LC and IP-RP-LC have been described. See, e.g., García-Alvarez-Coque, M. C. 2015. Reversed Phase Liquid Chromatography. In Analytical Separation Science; eds V. Pino, J. L.; doi.org/10.1002/9783527678129.assep008; Nwokeoji A. O. 2019. High resolution fingerprinting of single and double-stranded RNA using ion-pair reverse-phase chromatography. J Chromatogr B Analyt Technol Biomed Life Sci., 1104, 212-219, DOI: 10.1016/j.jchromb.2018.11.027.

In some embodiments, the LC process is RP-LC. In some embodiments, the RP-LC process comprises eluting the nucleosides from an RP-LC apparatus with a mobile phase solution comprising a mixture of a first solvent solution and a second solvent solution.

In some embodiments, the first solvent solution comprises one or more solvents selected from the group consisting of: acetonitrile; ammonium acetate; ethanol; formic acid; hexylene glycol; isopropanol; methanol; tetrahydrofuran; and water. In some embodiments, the first solvent solution comprises ammonium acetate and formic acid.

In some embodiments, the first solvent solution comprises about 1 mM to about 100 mM ammonium acetate, e.g., about 1 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 mM to about 100 mM ammonium acetate.

In some embodiments, the first solvent solution comprises about 0.01% to about 1.0% formic acid, e.g., about 0.01% to about 0.05%, 0.05% to about 0.1%, 0.1% to about 0.5%, or about 0.5% to about 1.0% formic acid.

In some embodiments, the first solvent solution comprises 20 mM ammonium acetate. In some embodiments, the first solvent solution comprises 0.1% formic acid. In some embodiments, the first solvent solution comprises 20 mM ammonium acetate and 0.1% formic acid.

In some embodiments, the second solvent solution comprises one or more solvents selected from the group consisting of: acetonitrile; ammonium acetate; ethanol; formic acid; hexylene glycol; isopropanol; methanol; tetrahydrofuran; and water. In some embodiments, the second solvent solution comprises methanol.

In some embodiments, the second solvent solution in the mobile phase solution mixture increases in concentration from about 1% to about 100% during the RP-LC process. In some embodiments, the concentration of the second solvent solution in the mobile phase solution mixture: a) increases from about 2% to about 21% over about 6.5 minutes; b) increases from about 21% to about 100% over about 0.45 minutes, and c) decreases from about 100% to about 0% immediately thereafter.

In another aspect of the present disclosure, the method further comprises an additional step of characterizing a sample of the separated double-stranded nucleic acids by an ion pair reverse phase liquid chromatography (IP-RP-LC) process. In some embodiments, the IP-RP-LC process comprises eluting the nucleosides from an IP-RP-LC apparatus with a mobile phase solution comprising a mixture of a first solvent solution and a second solvent solution. In some embodiments, the first solvent solution comprises one or more solvents selected from the group consisting of: acetonitrile; ammonium acetate; ethanol; formic acid; hexylene glycol; isopropanol; methanol; tetrahydrofuran; and water. In some embodiments, the first solvent solution comprises one or more ion pairs selected from the group consisting of: hexylammonium acetate; triethylammonium acetate; tetrabutylammonium phosphate; and dibutylammonium acetate.

In some embodiments, the concentration of each of the one or more ion pairs in the first solvent solution is from about 1 mM to about 100 mM, e.g., about 1 mM to about 5 mM, about 5 mM to about 10 mM, about 10 mM to about 15 mM, about 15 mM to about 20 mM, about 20 mM to about 25 mM, about 25 mM to about 30 mM, about 30 mM to about 35 mM, about 35 mM to about 40 mM, about 40 mM to about 45 mM, about 45 mM to about 50 mM, about 50 mM to about 55 mM, about 55 mM to about 60 mM, about 60 mM to about 65 mM, about 65 mM to about 70 mM, 70 mM to about 75 mM, about 75 mM to about 80 mM, 80 mM to about 85 mM, about 85 mM to about 90 mM, about 90 mM to about 95 mM, or about 95 mM to about 100 mM.

In some embodiments, the first solvent solution comprises 25 mM hexylammonium acetate.

In some embodiments, the second solvent solution comprises one or more solvents selected from the group consisting of: acetonitrile; ammonium acetate; ethanol; formic acid; hexylene glycol; isopropanol; methanol; tetrahydrofuran; and water. In some embodiments, the second solvent solution comprises acetonitrile.

In some embodiments, the second solvent solution in the mobile phase solution mixture increases in concentration from about 1% to about 100% during the IP-RP-LC process. In some embodiments, the concentration of the second solvent solution in the mobile phase solution mixture: a) remains at about 5% over about 2.5 minutes; b) increases from about 5% to about 50% over about 12.5 minutes; c) increases from about 40% to about 45% over about 5 minutes; d) increases from about 45% to about 80% over about 5 minutes; e) remains at about 80% for about 5 minutes, and f) decreases from about 80% to about 5% immediately thereafter.

It is also included within the present invention to detect and determine the amount of the RNA fragments itself, e.g. e.g., by determining the number of RNA fragments or the mass of the RNA fragments. Spectroscopic methods for RNA quantification include traditional absorbance measurements at 260 nm and more sensitive fluorescence techniques using fluorescent dyes such as ethidium bromide and a fluorometer with an excitation wavelength of 302 or 546 nm (Gallagher, 2011. Quantitation of DNA and RNA with Absorption and Fluorescence Spectroscopy. Current Protocols in Molecular Biology. 93: A.3D.1-A.3D.14).

A mass spectrometer (MS) is a gas phase spectrometer that measures a parameter that can be translated into mass-to-charge ratio of gas phase ions. Examples of mass spectrometers are time-of-flight, magnetic sector, quadrupole filter, ion trap, ion cyclotron resonance, electrostatic sector analyzer and hybrids of these. Methods for the application of MS methods to the characterization of nucleic acids are known in the art.

For example, Matrix-Assisted Laser Desorption/Ionization Mass Spectrometry (MALDI-MS) can be used to analyze oligonucleotides at the 120-mer level and below (Castleberry et al., 2008. Matrix-Assisted Laser Desorption/Ionization Time-of-Flight Mass Spectrometry of Oligonucleotides. Current Protocols in Nucleic Acid Chemistry. 33:10.1.1

Electrospray Ionization Mass Spectrometry (ESI-MS) allows the analysis of high-molecular-weight compounds through the generation of multiply charged ions in the gas phase and can be applied to molecular weight determination, sequencing and analysis of oligonucleotide mixtures (Castleberry et al., 2008. Electrospray Ionization Mass Spectrometry of Oligonucleotides. Current Protocols in Nucleic Acid Chemistry. 35:10.2.1-10.2.19). Preferably, the mass spectrometry analysis is conducted in a quantitative manner to determine the amount of RNA.

It is also included within the present invention to detect and determine the nucleic acid sequences of the separated double-stranded nucleic acids. Methods for sequencing of RNA are known in the art. A recently developed technique called RNA Sequencing (RNA-Seq) uses massively parallel sequencing to allow for example transcriptome analyses of genomes at a far higher resolution than is available with Sanger sequencing- and microarray-based methods. In the RNA-Seq method, complementary DNAs (cDNAs) generated from the RNA of interest are directly sequenced using next-generation sequencing technologies. RNA-Seq has been used successfully to precisely quantify transcript levels, confirm or revise previously annotated 5′ and 3′ ends of genes, and map exon/intron boundaries (Eminaga et al., 2013. Quantification of microRNA Expression with Next-Generation Sequencing. Current Protocols in Molecular Biology. 103:4.17.1-4.17.14). Consequently, the amount of the RNA fragments can be determined also by RNA sequencing.

### EXAMPLES

The following examples are meant to be illustrative and should not be construed as further limiting. The contents of the figures and all references, patents, and published patent applications cited throughout this application are expressly incorporated herein by reference.

### Example 1: Materials and Methods

### Primer and Plasmid

To produce the DNA templates for the in vitro transcription (IVT) reaction, the primers listed in Table 1 were used. The primers were reconstituted and diluted to a final concentration of 10 μM. To produce the RNA standards, the commercially available CMV-Cas9-2A-GFP plasmid from Sigma-Aldrich was used.

TABLE 1
List of used primers in this study.

| SEQ | | |
|---|---|---|
| ID | | |
| NO: | Primer Name | Sequence (5′ to 3′) |
| 1 | T7 fw | TAATACGACTCACTATAGGGAGACC |
| | | CAAG |
| 2 | 85bp_rev | CTGTACTTCTTGTCCATGGTGGCG |
| 3 | 85bp_T7_rev | TAATACGACTCACTATAGGGAGACG |
| | | CCACCATGGACAAGAAGTACAG |
| 4 | 502bp_rev | CTGTCCACCAGTTTCTTTCTCAGGT |
| | | G |
| 5 | 502bp_T7_ | TAATACGACTCACTATAGGGAGACT |
| | rev | GTCCACCAGTTTCTTTCTCAGGTG |
| 6 | 1043bp_rev | GTATCTCTTGATCATAGAGGCGCTC |
| | | AG |
| 7 | 1043bp_T7_ | TAATACGACTCACTATAGGGAGAGT |
| | rev | ATCTCTTGATCATAGAGGCGCTC |
| 8 | 2038bp_rev | GTGTATCTCCGCCGCTTCAG |
| 9 | 2038bp_T7_ | TAATACGACTCACTATAGGGAGAGT |
| | rev | GTATCTCCGCCGC |
| 10 | 4161bp_rev | CCAGCTGAGACAGGTCGATC |
| 11 | 4161bp_T7_ | TAATACGACTCACTATAGGGAGACC |
| | rev | AGCTGAGACAGGTCG |

### Construction of DNA Templates

The DNA templates for the IVT reaction were produced by two amplification steps using primers designed to generate RNA constructs with different lengths. The second amplification with the PCR product from the first PCR was only performed to add a T7 promotor on both ends for the production of double-stranded RNA (dsRNA). The DNA constructs were amplified using the Q5® High-Fidelity 2X Master Mix from New England Biolabs. The annealing temperatures were calculated using the NEB Tm Calculator. For the amplification, the composition and thermocycling conditions were chosen based on the NEB protocol.

For the purification of the DNA constructs agarose gel electrophoresis was performed using precast agarose (1-4%) E-Gels with SYBR safe gel stain from Invitrogen. The gel extraction was performed using the NucleoSpin Gel and PCR Clean-up kit from MACHEREY-NAGEL. The DNA was quantified at 260 nm using the Thermo Scientific™ NanoDrop™ One©.

In Vitro Transcription dsRNA

To produce dsRNA standards, the MEGAscript RNAi Kit from Thermo Fisher Scientific was used. The transcription reaction was setup and performed according to the kit protocol using the generated DNA construct as templates. The reaction was stopped after an incubation for 4 hours followed by an RNA annealing step at 75° C. for 5 minutes. The removal of DNA and ssRNA by digestion with RNase and DNase was performed according to the kit protocol. For the purification of the dsRNA standards the solutions and columns provided in the kit were used. The elution of the dsRNA was done 2×80 μL with 65° C. pre-warmed elution solution.

The dsRNA standards were additionally purified by agarose gel electrophoresis and gel extraction using precast agarose (1-4%) E-Gels with SYBR safe gel stain from Invitrogen and the gel extraction was performed using the NucleoSpin Gel and PCR Clean-up kit from MACHEREY-NAGEL. To improve the recovery of the RNA the NTC buffer from MACHEREY-NAGEL was used to dissolve the gel pieces. The quality of dsRNA standards were checked by IP-RP-LC analysis. The concentrations were determined by total digestion to nucleosides and quantification analyses by RP-LC.

In Vitro Transcription ssRNA

To produce ssRNA standard, the MEGAscript T7 Transcription Kit from Thermo Fisher Scientific was used. The transcription reaction was setup and performed according to the kit protocol using the generated DNA construct as templates. The reaction was stopped after a 4-hour incubation. To remove the DNA template a digestion with TURBO DNase was performed according to the kit protocol. To purify the ssRNA a lithium chloride precipitation was performed according to the kit protocol. The RNA was resuspended in 500 μL RNase-free water. The quality of ssRNA standards were checked by IP-RP-LC analysis. The concentrations were determined by total digestion to nucleosides and analysis by RP-LC.

dsRNA Enzyme-Linked Immunosorbent Assay (ELISA)

The dsRNA sandwich ELISA was prepared with the anti-dsRNA antibody J2 as a coating antibody and K2 anti-dsRNA antibody (Jena BioScience) as the detection antibody. The J2 antibodies were immobilized on a 96-well MaxiSorp (white) at 4° C. overnight. The plates were blocked with 1% bovine serum albumen (BSA) in phosphate-buffered saline (PBS) for 1 hour at 25° C. The plates were washed, and the RNA samples were added and incubated for 2 hours at 25° C. After sample incubation, the plates were washed, and the anti-dsRNA antibody K2 was added and incubated for 1 hour at 25° C. The plates were washed followed by the addition of alkaline phosphatase-conjugated goat anti-Mouse IgM (u-Chain) detection antibody (Sigma-Aldrich) and incubating for 1 hour at 25° C. After washing the plates, the substrate PhosphaGLO AP (Seracare) was added and incubated at 25° C. for 1 hour. Chemiluminescence signal was detected at 450 nm on a SpectraMax i3X (Molecular devices) reader. dsRNA standard curves (1000 bp dsRNA (self-made) or 142 bp dsRNA from Jena BioScience) were generated and used for calculating and determining the dsRNA concentration.

### Chromatography Analyses

Native and RNase I_{f} (recombinant protein fusion of RNase I and maltose-binding protein) digested samples were analyzed using ion pair reverse phase (IP-RP-) chromatography on an Agilent 1290 Series. For the analysis, a ProSwift™ RP-1S (4.6×50 mm SS, Thermo Scientific) column was used. Signal detection was performed using a diode array detector (DAD) detector at a wavelength of 260 nm. The analysis was conducted under the following conditions: mobile phase A-25 mM Hexylammonium acetate, mobile phase B-Acetonitrile. The samples were analyzed with a flow rate of 1.2 mL/min at 40° C. using the following gradient: Starting at 5% B for 2.5 mins, increasing from 5% to 40% B over 12.5 mins, increasing from 40% to 45% B over 5 mins, increasing from 45% to 80% B over 5 mins, holding at 80% B for 2.5 mins, immediate change to 5% B. The sample tray was cooled to 5° C.

Nucleoside digested samples were analyzed using reverse phase (RP-) chromatography on an Agilent 1290 Series. For the analysis, an Atlantis™ T3 5 μm (4.6×50 mm, Waters) column was used. Signal detection was performed using a DAD detector at a wavelength of 260 nm. The analysis was conducted under the following conditions: mobile phase A-20 mM Ammonium acetate+0.1% formic acid, mobile phase B-Methanol. The samples were analyzed with a flow rate of 0.75 mL/min at 35° C. using the following gradient: 2% to 21% B over 6.5 mins, increasing from 21% to 100% B immediately, holding for 0.45 min, decreasing from 100% to 2% B immediately. The sample tray was cooled to 5° C.

### Standard Curve and Method Validation

Fifteen (15) standards were used to determine a calibration curve as an underlying for calculations in this work, applying apex integration algorithm. A 2.5 mM solution for each nucleoside (Adenosine [A], Cytidine [C], Uridine [U], and Guanosine [G]) was prepared by diluting the necessary stocks. From these 2.5 mM solutions, we prepared the following dilutions, each containing the four nucleosides: 1.25 mM, 1 mM, 750 μM, 500 μM, 250 μM, 100 μM, 50 μM, 25 μM, 10 μM, 5 μM, 2.5 μM, 1 μM, 500 nM, 250 nM, 100 nM, and 50 nM. The same procedure was applied to the deoxynucleotides. Standards were prepared in triplicates. All standards were analyzed using the RP-chromatography method. The injection volume for each standard was 5 μL. The determined relative retention times (RRT) values relative to adenosine are listed in Table 2.

TABLE 2
Determined relative retention times (RRT) values of nucleosides.

| Nucleoside | RRT (vs dA) [%] |
|---|---|
| Cytidine (C) | 0.29 |
| Deoxycytidine (dC) | 0.38 |
| Uridine (U) | 0.42 |
| 1N-Methylpseudouridine (PU) | 0.43 |
| Guanosine (G) | 0.68 |
| Deoxyguanosine (dG) | 0.76 |
| Thymidine (T) | 0.84 |
| Adenosine (A) | 0.94 |
| Deoxyadenosine (dA) | 1.00 |

### Sample Preparation and Analyses

Double-stranded RNA samples were diluted to 50 ng/μL working solution. 10 μL of the working solution was mixed with 2 μL RNase I_{f} (5 U/μL), 1.8 μL NEBuffer 3 (New England Biolabs) and 6.2 μL RNase-free water. The sample was incubated for 3.5 h at 37° C. 30 μL RNase-free water was added to it. The purification was performed according to the manufacturer protocol of the Monarch PCR & DNA Cleanup Kit (New England Biolabs). The bound dsRNA was eluted twice using 20 μL RNase-free water. 17 μL of the eluted sample was transferred into a new tube and mixed with 1 μL Nucleoside Digestion Mix and 2 μL Nucleoside Digestion Mix buffer (New England Biolabs). Sample was then incubated for 2 h at 37° C. and used for nucleoside analysis via RP Chromatography. 20 μL of the eluted sample was used for IP-RP-Chromatography. Samples were prepared in quadruplicates, where 10 μL of the working solution was diluted with 30 μL RNase-free water (reference sample solution). 17 μL of the reference sample solution was transferred into a new tube and mixed with 1 μL Nucleoside Digestion Mix and 2 μL Nucleoside Digestion Mix buffer. The sample was incubated for 2 h at 37° C. and used for the nucleoside analysis via RP-chromatography. The remaining reference sample solution was used for the IP-RP-chromatography.

11 μg mRNA (EGFP, firefly luciferase (FLuc) and Cas9; TriLink BioTechnologies (San Diego, USA)) sample was incubated with 4.4 μL RNase I_{f} (50 U/μL), 8.8 μL NEBuffer 3 and 63.8 μL RNase-free water for 3.5 h at 37° C. Purification was performed according to the manufacturer procedure of the Monarch PCR & DNA Cleanup Kit (New England Biolabs). Elution of bound dsRNA was managed two times with 10 μL RNase-free water. 17 μL of eluted sample was transferred into a new tube. 2 μL nucleoside digestion buffer and 1 μL nucleoside digestion mix was added. The solution was incubated for 2 h at 37° C. The samples were analyzed via RP-Chromatography. Identical sample preparation was performed for IP-RP-chromatography but without the second digestion step. All samples were prepared in a fourfold manner. Injection volume for RP-chromatography and IP-RP-Chromatography were 15 μL and 16 μL, respectively.

### Result Method Validation RP-LC

The linearity was determined for each (deoxy-) nucleoside in the concentration range of 50 nM-1.25 mM. Linearity was demonstrated throughout the entire range with determination coefficients R2≥0.999. In addition, accuracy, precision, and LOD/LOQ were calculated from the used standards. Injection precision was determined by a six-fold injection. LOD and LOQ correspond to the 3*S/N respectively 10*S/N ratio. For determination of accuracy, method precision (high and low concentrations with three individual injections) as well as injection precision (six individual injections) of (deoxy-) nucleosides, the linearity standards were used for determination and calculation of respective values (Table 4).

### Calculations

Calculations are based on the following equations.

### Recovery Purification:

$${R}_{IP} = \frac{{A}_{Purified}}{{A}_{Native}} * 100$$

R_{IP}=Recovery IP-chromatography

$$A = Area [ {AU}^{*} s ]$$

Amount (Deoxy-) Nucleotide after NDM:

$${m}_{n u c} = \frac{{A}_{n u c} * {M}_{x} * {V}_{IV 1} * {V}_{1} * {V}_{3}}{{S}_{n u c} * 1000 * {V}_{IV 2} * {V}_{2}}$$

m_{nuc}=Amount (deoxy-) nucleoside [μg]
A_{nuc}=Area (deoxy-) nucleoside [AU*s]
S_{nuc}=Slope of individual (deoxy-) nucleoside linearity [AU*s/mM]
Mₓ=Molecular weight Oxygen corrected [g/mol]
V_{IV1}=Injection volume standard 5 μL
V_{IV2}=Injection volume sample 15 μL
V₁=Volume Nucleoside Digestion 20 μL
V₂=Eluted sample volume used for nucleoside digestion 17 μL
V₃=Eluted sample volume 40 μL

For the calculation of the nucleotide amounts, a modified molecular weight as an approximation was used. This is because the RNA molecule has one less oxygen atom in the phosphate backbone compared to its nucleoside building blocks. Molecular weights used for this calculation are given in Table 3.

TABLE 3
Modified molecular weights used for
nucleotide content calculation.

| Nucleotide | MW [g/mol] | Deoxynucleotide | MW [g/mol] |
|---|---|---|---|
| Cytidine | 307.19 | Deoxycytidine | 291.20 |
| Guanosine | 347.22 | Deoxyguanosine | 331.22 |
| Uridine | 308.18 | Thymidine | 304.19 |
| Adenosine | 331.22 | Deoxyadenosine | 315.22 |
| m1Ψ | 322.20 | | |

Amount of dsRNA/dsDNA:

$${m}_{dsRNA} = {m}_{C} + {m}_{U} + {m}_{G} + {m}_{A}$$

m_{dsRNA}=Amount of dsRNA [μg]
m_{C}=Amount Cytidine [μg]
m_{U}=Amount Uridine or m1Ψ [μg]
m_{G}=Amount Guanosine [μg]
m_{A}=Amount Adenosine [μg]

$${m}_{dsDNA} = {m}_{d C} + {m}_{d G} + {m}_{T} + {m}_{d A}$$

m_{dsDNA}=Amount of dsDNA [μg]
m_{dC}=Amount Deoxycytidine [μg]
m_{dG}=Amount Deoxyguanosine [μg]
m_{T}=Amount Thimidine [μg]
m_{dA}=Amount Deoxyadenosine [μg]

### Total Recovery:

$${R}_{T R} = \frac{{m}_{dsRNA}}{{m}_{R e f}} * 100$$

R_{TR}=Total recovery [%]
m_{dsRNA}=Amount dsRNA after purification and digestion [μg]
m_{Ref}=Amount dsRNA of reference sample solution [μg]

### Efficiency Digestion Reaction:

$${E}_{D R} = \frac{{R}_{T R} * 100}{{R}_{IP}}$$

E_{DR}=Efficiency of the digestion reaction [%]

### Example 2: Methodology Design and Workflow

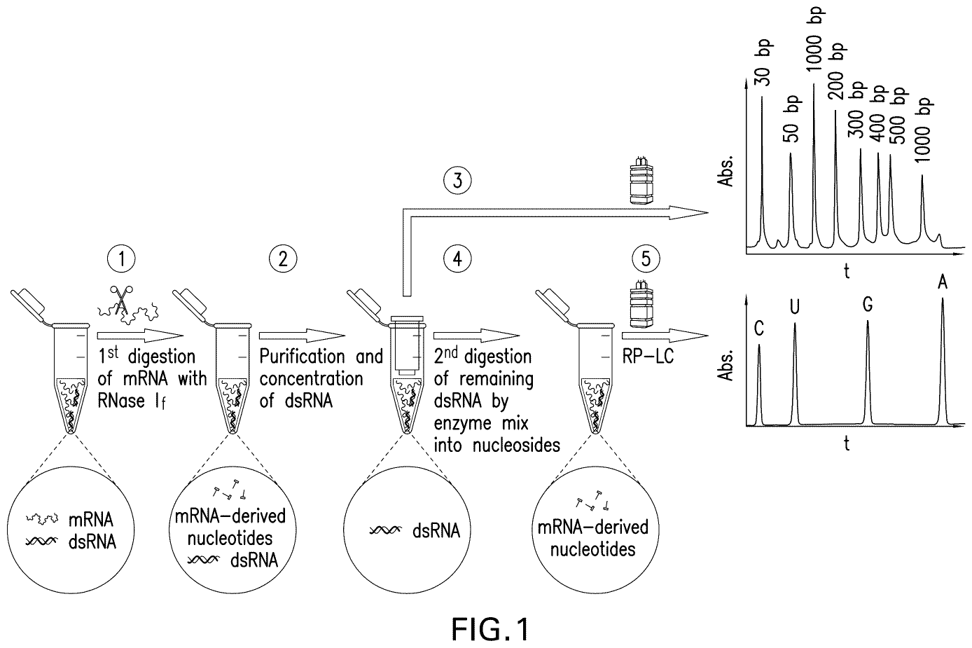

For the accurate quantification and description of dsRNA contamination in mRNA therapeutics, a full methodology was developed that involved two chromatography methods, both preceded by sample preparation and purification steps. An overview of the procedure including the steps involved is shown in FIG. 1.

In the initial stage, to target and isolate the dsRNA population specifically, an enzymatic digestion step was employed, using RNase I_{f} (recombinant protein fusion of RNase I and maltose-binding protein) to degrade all mRNA, while leaving polynucleotides composed of dsRNA and eventually some in vitro transcription-derived remaining dsDNA behind (FIG. 1, step 1).

Impurities, monomers, and enzymes were removed, while maintaining high recovery rates for both dsRNA and dsDNA, and while preserving the integrity of the targeted polynucleotides using the Monarch PCR & DNA Cleanup Kit from NEB (FIG. 1, step 2).

For the downstream chromatography analyses, two distinct approaches were implemented. The first method, an ion pair reverse phase liquid Chromatography (IP-RP-LC), served as a control measure for sample preparation and qualitative characterization of dsRNA populations as described in Nwokeoji et al., (2017) J Chromatogr A. 1484, 14-25 and Nwokeoji et al., (2019) J Chromatogr B Analyt Technol Biomed Life Sci. 1104, 212-219. By optimizing the chromatographic conditions with the use of hexylammonium acetate (HAA) as an ion-pairing agent in the mobile phase (Donegan et al., (2022) J Chromatogr A. 1666, 462860), the effective separation of various dsRNA species was achieved, enabling visualization of polynucleotides from 20 bp up to more than 4000 bp in a single run (FIG. 1, step 3).

The second chromatography method (RP-LC) was applied to quantify dsRNA and dsDNA via its monomers. A second enzymatic digestion was performed using an enzyme cocktail, named nucleoside digestion mix (NDM; Crippen et al., (2019) J Virol. 93 (23), e01111-19), which effectively degraded both RNA and DNA polynucleotides into nucleosides in a one-step digestion (FIG. 1, step 4). Subsequent analysis of the nucleosides was performed to accurately quantify the dsRNA and remnant dsDNA content (FIG. 1, step 5).

### Example 3: IP-RP-LC Implementation

To effectively separate and characterize polynucleotides, with a focused intent on the small to medium-sized dsRNA fragments, the IP-RP-LC method was developed. An effective separation of the polynucleotides and a satisfactory signal-to-noise ratio was achieved through the implementation of the hydrophobic ion pair agent hexylammonium acetate (HAA).

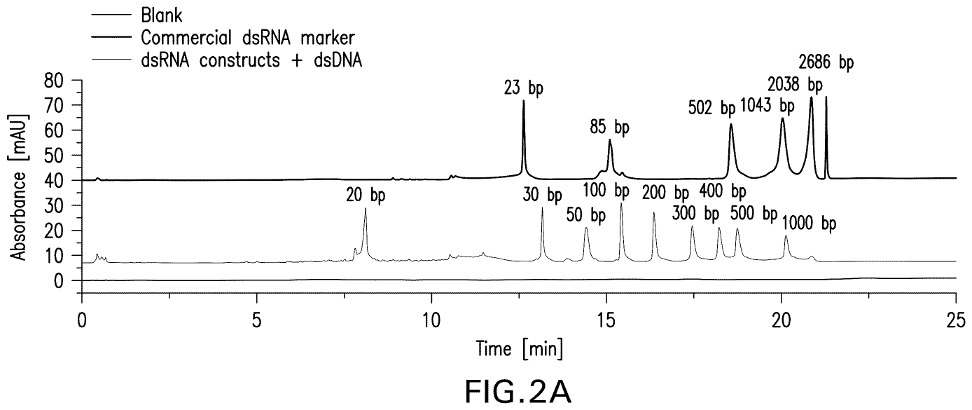

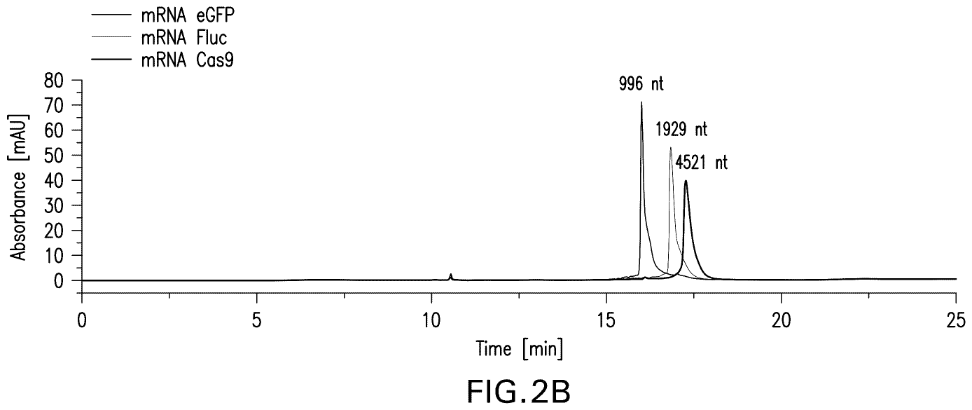

The principal focus was on achieving optimal resolution for dsRNA fragments (FIG. 2A). A commercially available dsRNA size marker was used, while also using custom generated dsRNA constructs of defined lengths (85 bp, 502 bp, 1043 bp, 2038 bp, and 4161 bp) through T7 transcription reactions. The 85 bp-2038 bp constructs, in conjunction with a synthetic 23 bp dsRNA construct and a circular dsDNA plasmid (2686 bp), were effectively separated using the annotated method (FIG. 2A). Furthermore, the developed IP-RP-LC method demonstrated the ability to also separate single-stranded mRNA constructs (ssRNA) according to their respective sizes (FIG. 2B). The used mRNA constructs encoding Fluc, eGFP, and Cas9 were obtained commercially and served as surrogate samples. Noteworthy, using the same method, the elution behavior of dsRNA and mRNA species of similar lengths significantly differed from each other.

In this study, the IP-RP-LC method was used for the qualitative determination and description of polynucleotide populations, aiding in the comparison of dsRNA peak signals and the evaluation of purification process efficiency, by identifying potential losses. Moreover, both commercially acquired and custom-made dsRNA constructs were evaluated for purity through the utilization of the IP-RP-LC method.

### Example 4: RP-LC Implementation

The RP-LC method was implemented for the purpose of quantifying polynucleotides based on detected monomers. It was used to accurately quantify low nucleoside and deoxynucleosides levels. This allowed the back-calculation of dsRNA and residual dsDNA quantities in tested samples. N1-Methyl-Pseudouridine (m1Ψ) was included into method development and characterization. The performance parameters were validated as demonstrated in Table 4. Table 4 summarizes the validated parameters, grouped for four nucleosides and five deoxynucleosides. The parameters were determined in triplicate.

TABLE 4
Summary of validated RP-LC analytical range.

| | | | Method | | Injection | |
|---|---|---|---|---|---|---|
| | LOD | LOQ | Precision | Accuracy | Precision | |
| Samples | [pg] | [pg] | [%] | [%] | [%] | Linearity |
| Nucleosides | 45-73 | 150-244 | 0.5-3.6 | 96-100 | 0.1-0.3 | 50 nM-1.25 mM |
| A, U, m1Ψ, G and C | | | | | | with each determination |
| Deoxynucleosides | 48-78 | 160-260 | 0.3-0.9 | 97-100 | 0.3-0.9 | coefficient |
| dA, T, dG and dC | | | | | | R² ≥ 0.999 |

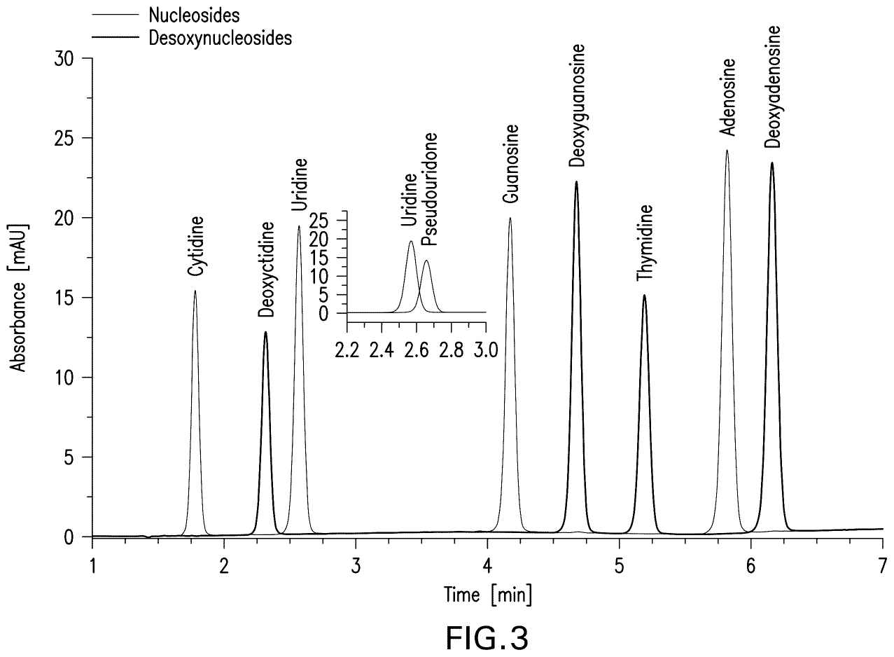

The developed method enabled a nucleoside quantification at picogram levels, having baseline separated peaks at analytically significant signal-to-noise values (FIG. 3). Beside determination of isolated dsRNA quantities, this method was further utilized to determine the concentration of ssRNA and dsRNA reference material.

### Example 5: Efficiency and Recovery Performance of the Method

The efficiency and recovery performance of the method were examined by analyzing the effects of both enzymatic digestions as well as the purification step, conducted prior to chromatographic quantification. Minimal amounts of dsRNA constructs (approx. 300 ng) with sizes of 23 bp, 85 bp, 502 bp, 1043 bp, 2038 bp, and 4161 bp were utilized, along with a circular dsDNA construct of 2686 bp to simulate the broad size range of potential double-stranded polynucleotide contaminants in in vitro transcription-derived mRNA products.

Total recovery consisted of two parts: the recovery after the initial enzymatic digestion and purification, and the efficiency of the subsequent enzymatic nucleoside digestion step (FIG. 1). This total recovery, as determined for the respective constructs, reflected the general efficiency of the method. For reference values, the starting material was digested into its monomers using the NDM enzyme cocktail and quantified via RP-LC.

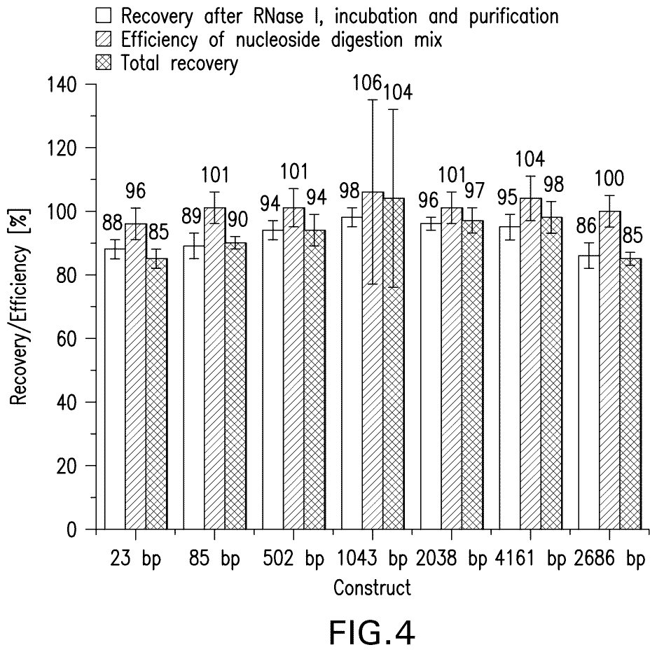

During the first step, the constructs underwent incubation with RNase I_{f}. This enzyme is known in the art for its high preference for single-stranded RNA over double-stranded RNA, with the ability to cut ssRNA between any phosphodiester bond. RNase I can bind to DNA but does not digest it as a substrate (Kennell (2002) J Bacteriol. 184 (17), Wang et al., (2018) BMC Biotechnol. 18 (1), 1-12). Efficiency of the RNase I_{f} incubation and the subsequent purification was determined by comparing the peak area of digested and purified samples with untreated reference samples. As demonstrated in FIG. 4, only minimal losses were documented. For the dsRNA constructs, efficiency increased with the size of the constructs, and the loss from the combined RNase I_{f} digestion and purification steps averaged between 2% and 12% for dsRNA and 14% for dsDNA (first set of bars, FIG. 4).

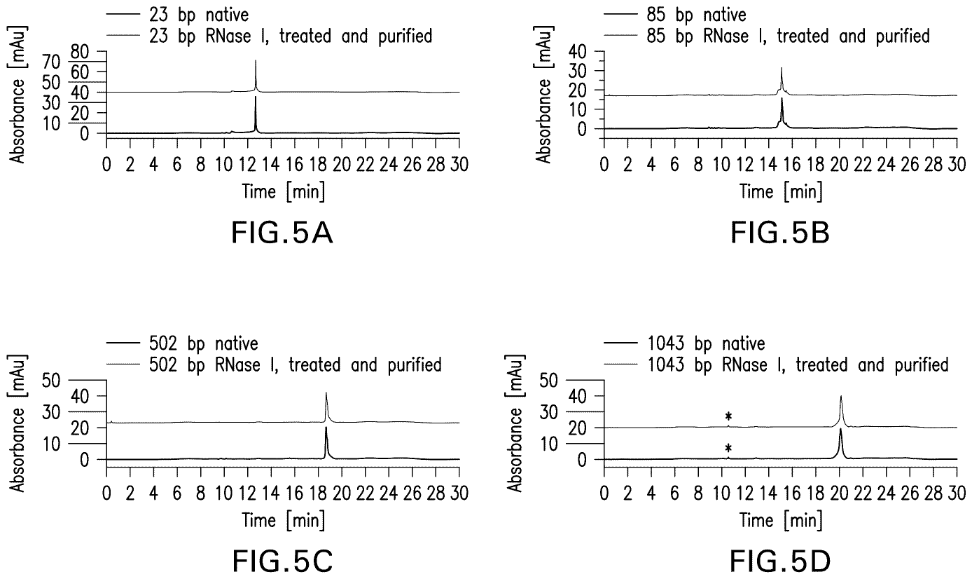

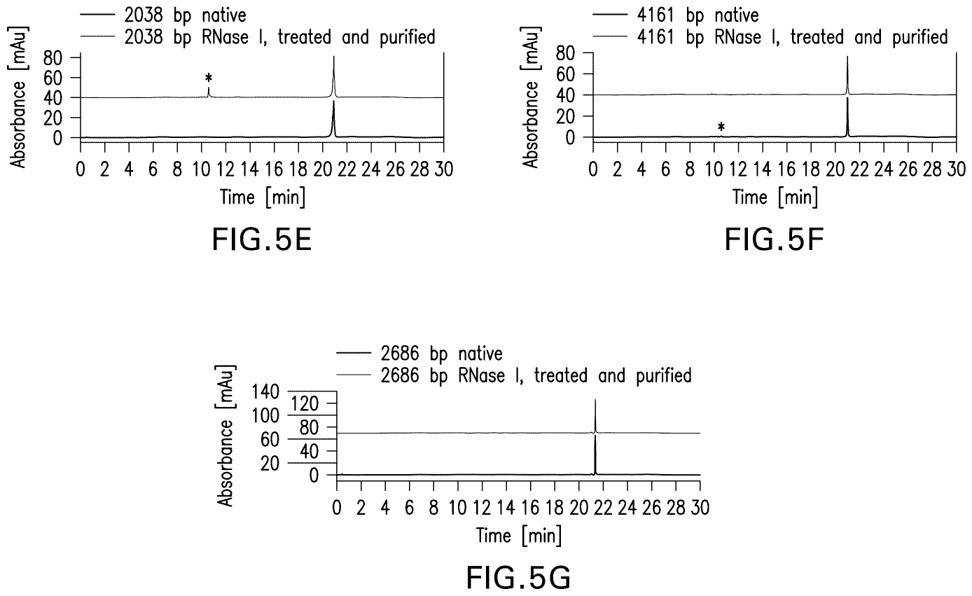

The final, total recovery (third set of bars, FIG. 4) was quantified by further digesting the purified constructs into nucleoside monomers using NDM and performing quantification through RP-LC. These obtained values were based on a comparison against the reference values of the untreated starting material, which was also digested to nucleosides using the NDM enzyme cocktail. Following adjustment of the initial reference values to cater for the efficiency previously identified in the first step, the efficiency of the NDM-mediated step could be isolated (middle set of bars, FIG. 4). This second enzyme step was highly efficient for all constructs used, with only a further loss of about 4% documented for the smallest dsRNA construct of 23 bp. For all the remaining constructs, on average, the conversion was complete. Consequently, the total recovery was mainly influenced by the first RNase I_{f} incubation with the associated cleaning process. Furthermore, the integrity of dsRNA peaks in the chromatograms was not compromised (FIG. 5). Under the established conditions with a maximal enzyme to substrate ratio of 20 units per microgram substrate up to 210 minutes incubation, no indication of dsRNA degradation by RNase I_{f} was found.

To validate the recovery performance of the methodology in an mRNA sample background, a dsRNA construct of 502 bp was spiked into an mRNA sample matrix composed of 1000 nucleotides. The recovery of spiked dsRNA was evaluated at the three different spike levels 0.03%, 0.3%, and 3% in quadruplicates. The dsRNA recovery determined across three logarithmic scales of spiking levels ranged from 87% to 104%.

### Example 6: Description and Quantification of dsRNA Populations in mRNA Samples

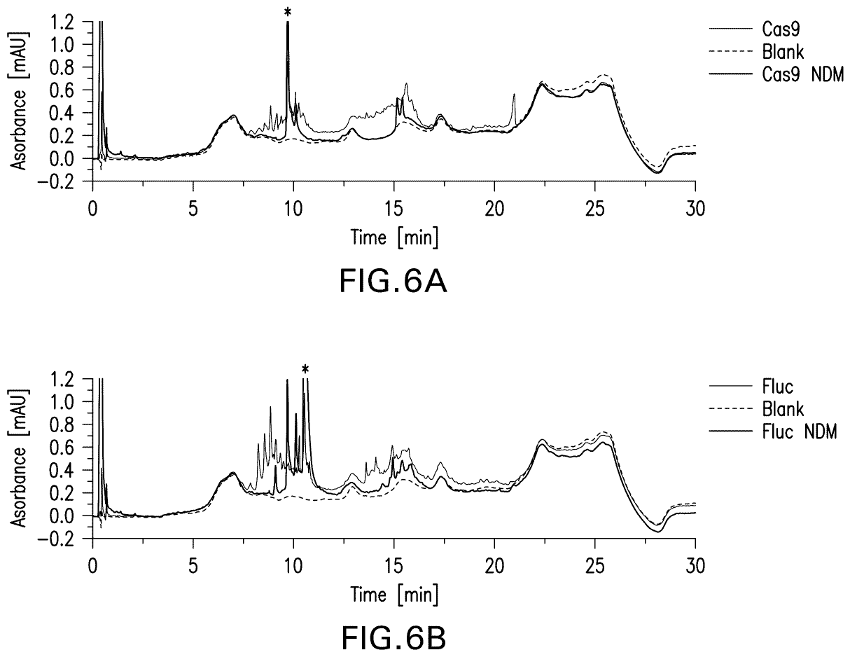

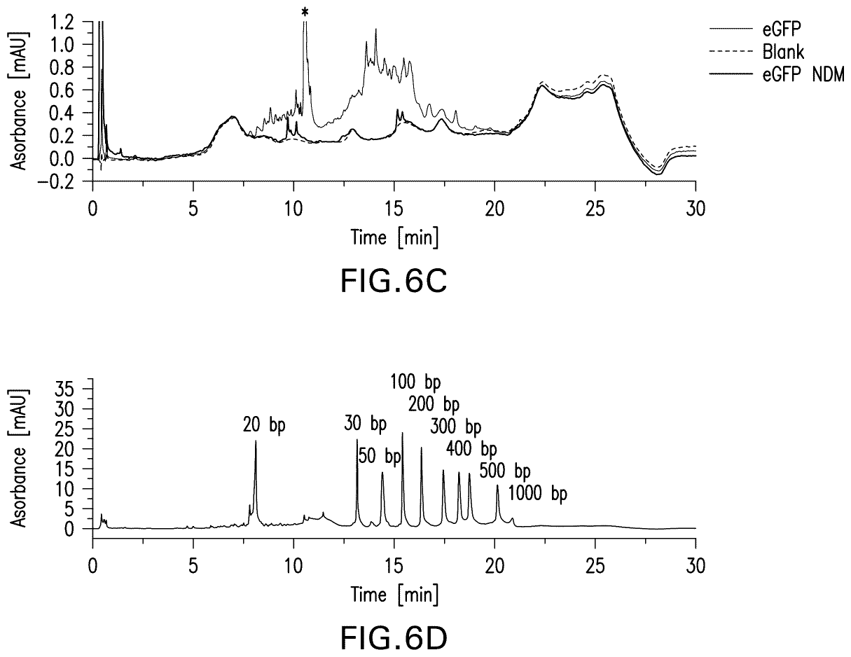

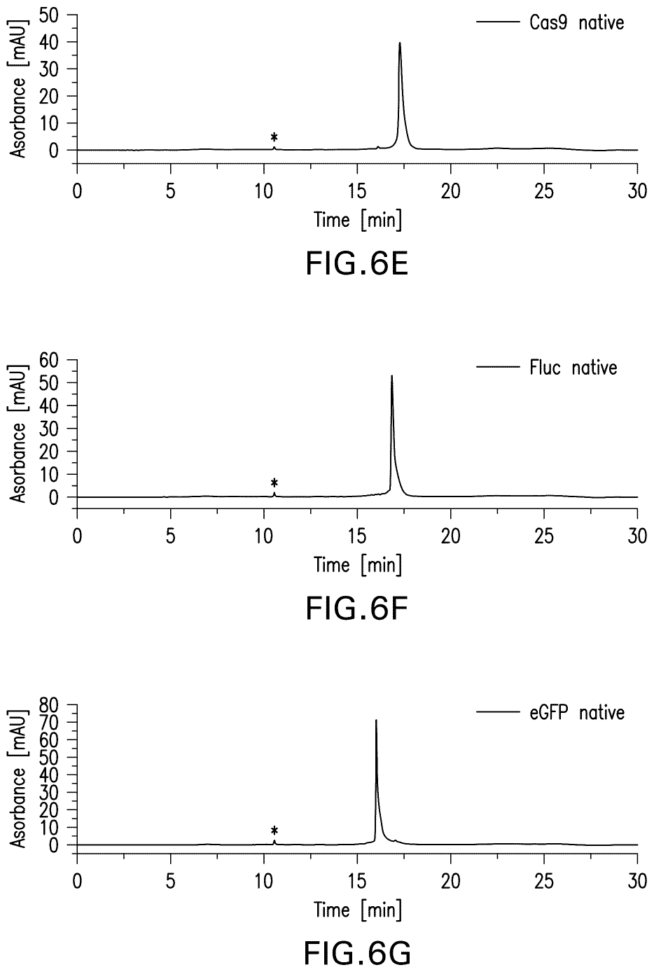

Three surrogate samples intended to mimic vaccine drug substances were procured. These included in vitro transcription (IVT)-derived mRNA constructs encoding for eGFP, firefly luciferase (Fluc), and Cas9. The surrogate samples varied in size, specifically 996 nt for eGFP, 1929 nt for Fluc and 4521 nt for Cas9. Each mRNA sample was individually analyzed using IP-RP-LC to determine the retention time of each construct (FIGS. 6E-6G). To display the dsRNA population of the three constructs, a quantity of eleven micrograms was subjected to RNase I_{f} digestion, purified and analyzed by IP-RP-LC (FIGS. 6A-6C, gray traces). To verify the detected signals after RNase I_{f} digestion can be assigned to dsRNA species, the samples were further digested with NDM and analyzed by IP-RP-LC (FIGS. 6A-6C, black traces).

The comparison of the chromatograms of the native constructs with the RNase I_{f} digested ones revealed that all the single-stranded mRNA constructs were completely digested, and no significant signals were recorded at the respective retention times of the three mRNA peaks.

Involving a dsRNA size standard (FIG. 6D), the isolated dsRNA populations depicted via the IP-RP-LC method were classified into three groups into small-sized (<30 bp), medium-sized (30-500 bp) and large-sized (>500 bp) species. A significant fraction of the isolated polynucleotides in the mRNA constructs appeared to be small-sized species. The medium-sized dsRNA population seemed to predominate in the eGFP sample, but a notable quantity of those polynucleotides was also detected in the Fluc and Cas9 samples. Neither eGFP nor Fluc samples exhibited signals of large-sized species. However, the Cas9 sample showed a distinct, large-sized dsRNA population larger than 1000 bp.

Comparing the overlayed chromatograms of the RNase I_{f} digested and NDM digested mRNA constructs (FIGS. 6A-6C), it was observed that most of the signals detected after RNase I_{f} digestion could be assigned to dsRNA and/or dsDNA related species since the signals disappeared after NDM digestion. Especially the black trace in the overlay from Cas9 (FIG. 6A) and eGFP (FIG. 6C) showed only a few low intensity peaks after total digestion. Since the remaining peaks were not digested by the NDM the signals could be assigned to non-polynucleotide related species.

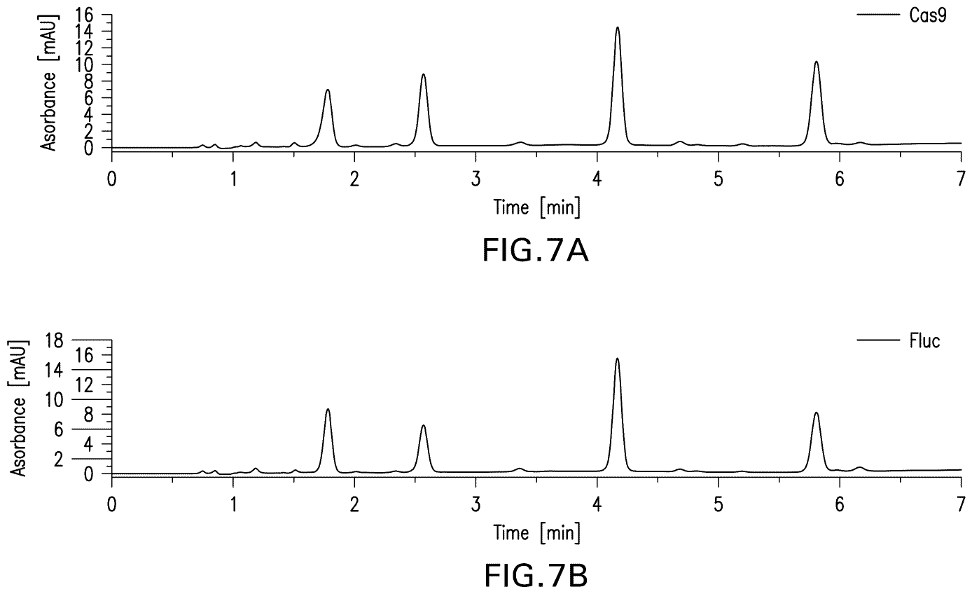

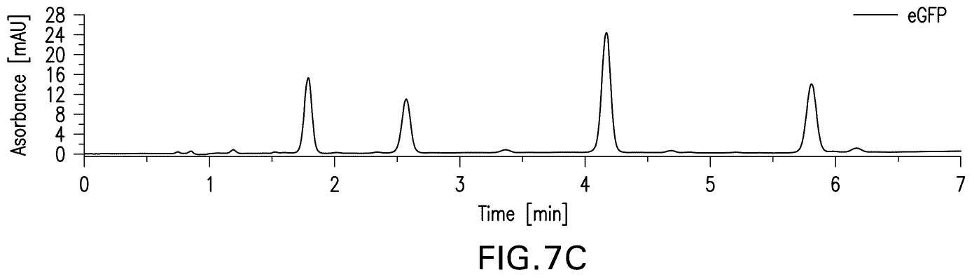

Besides the characterization of the dsRNA species by IP-RP-LC, the dsRNA and dsDNA concentrations for each mRNA construct were determined in quadruplicates based on nucleoside level by RP-LC. The findings from these evaluations were subsequently compared to the results obtained in an ELISA assay of the three corresponding mRNA samples. All results from the quantification by RP-LC and ELISA are summarized in Table 5, representative RP-LC chromatograms used for quantification are shown in FIG. 7. Table 5 demonstrated a summary of determined dsRNA concentration levels (w/w) in commercial mRNA surrogate samples by RP-LC and ELISA. The RP-LC-based quantification assay was also used to determine additional dsDNA contamination levels (w/w). Samples analyzed by ELISA were tested in triplicates and samples quantified by RP-LC quadruplicates.

TABLE 5
dsRNA quantification results based on ELISA and RP-LC Assay.

| | ELISA Assay | | RP-LC Assay | | | |
|---|---|---|---|---|---|---|
| | dsRNA | SD | dsRNA | SD | dsDNA | SD |
| Sample | [%] | [%] | [%] | [%] | [%] | [%] |
| Cas9 | 2.32 | 0.10 | 1.20 | 0.11 | 0.04 | <0.01 |
| Fluc | 1.56 | 0.12 | 1.24 | 0.02 | 0.03 | <0.01 |
| eGFP | 2.59 | 0.21 | 2.07 | 0.09 | 0.04 | <0.01 |

For two out of three samples, the RP-LC determined dsRNA values were within the range of those determined by ELISA. However, notably, the ELISA data were consistently higher. Particularly in the case of the Cas9 sample, the ELISA data indicator was significantly elevated, by more than 90% (See, Example 7 for direct comparison of these two detection methods). Noteworthy, alongside dsRNA determination, a value of 0.03%-0.04% residual dsDNA was detected in all three samples using the implemented procedure.

### Example 7: Comparison of Detection Methods: ELISA and LC

A critical comparison between the dsRNA detection methods of ELISA and LC revealed inherent limitations of ELISA and advantages of LC. The inability of an ELISA assay, using the J2 and K2 antibody combination, to detect small dsDNA fragments is due to these antibodies' specific binding motif of dsRNA. The binding to dsRNA occurs from a size of 40 base pairs and certain sequences presenting adenosine residues on the surface can enhance this binding (Schönborn et al., (1991) Nucleic Acids Res. 19 (11), 2993-3000, Bonin et al., (2000) RNA. 6 (4), 563-570). This showed a disadvantage of immunoassays and explained why the ELISA-obtained value for the Cas9 sample was higher than the LC-obtained comparative value.

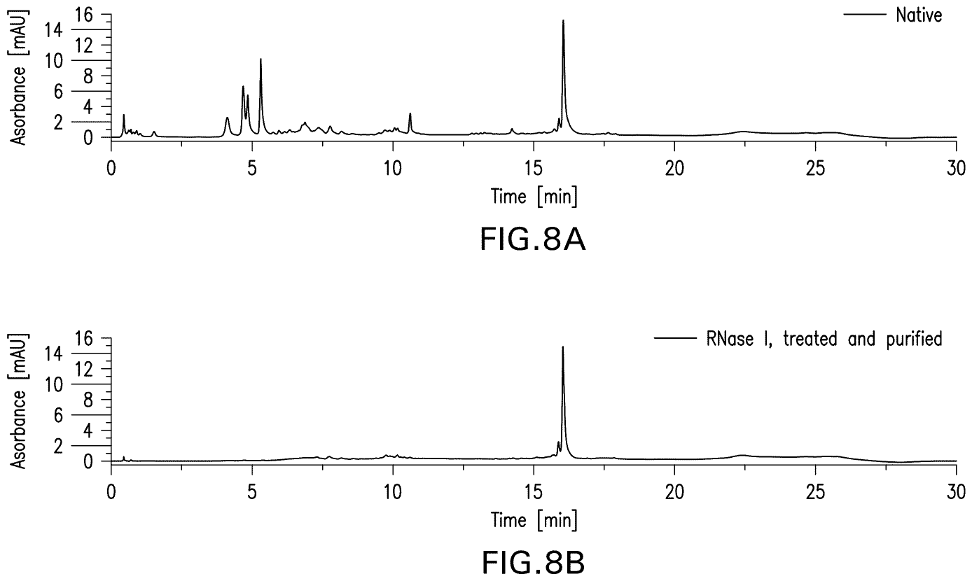

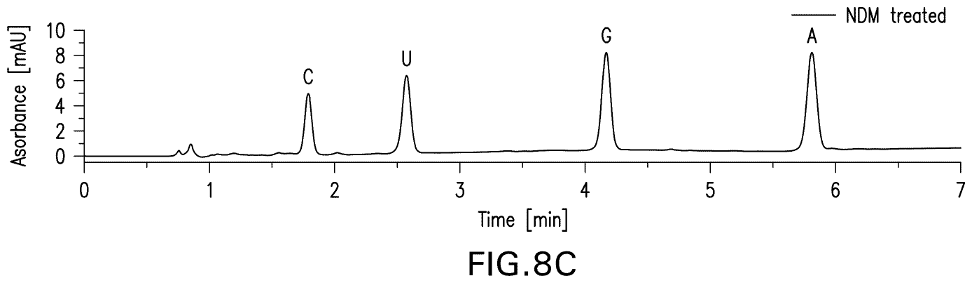

A further but related drawback of the ELISA assay is that the obtained values are based on a reference standard value, making the results dependent on the standard used. The current US Pharmacopeia draft recommends the use of a 142 bp dsRNA standard (US Pharmacopeia, USP. Analytical Procedures for MRNA Vaccine Quality, 2022). Accordingly, a standard of this size was sourced and used herein. By using the described nucleoside digestion approach, the concentration label claims for the obtained standard lots were verified and within a range of 95% to 100%. However, the IP-RP-LC profile revealed additional species of varied sizes, in addition to the expected main peak (FIG. 8A). Those populations were not composed of double-stranded dsRNA and were not detectable anymore after RNase I_{f} digestion, followed by subsequent purification (FIG. 8B). Consequently, after RNase I_{f} digestion and silica column preparation, the determined dsRNA concentration of the commercial standard dropped down up to 50% of the label claim for some lots. We noted comparable results for other commercially sourced standard lots and observed that resulting assay values can be influenced (data not shown). This observation led to the generation of in-house reference constructs used herein with the described sizes ranging from 85 bp to 4161 bp.

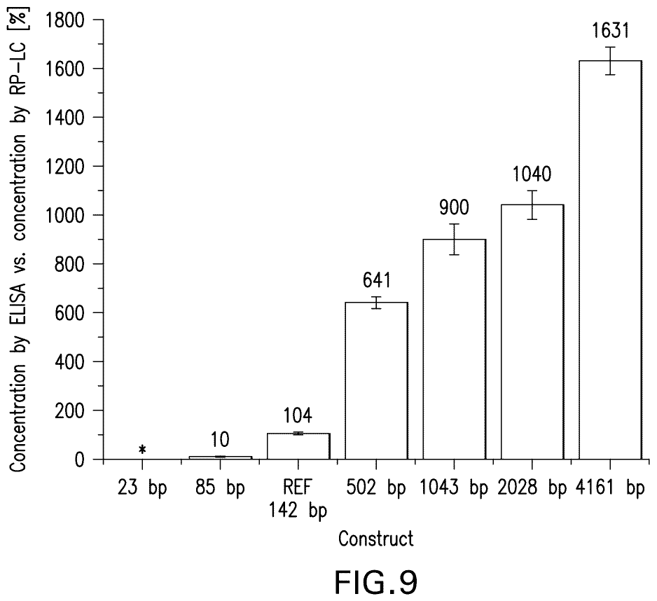

Each of the custom-made constructs were tested by an ELISA assay using the previously described 142 bp standard as a reference. As shown in FIG. 9, results confirmed that the J2 antibody required a minimum size for binding. Consequently, detection for the 23 bp construct was not possible with the conducted immunoassay. For the larger 85 bp construct, a ten times lower signal was detected on average. The opposite was observed for the larger constructs (502 bp to 4161 bp), where, relative to the 142 bp reference value, the ELISA signals and thus calculated concentrations were significantly higher. With increasing size of the dsRNA constructs, the response was getting stronger and peaked at an intensity more than sixteen-fold greater than the reference. The results distinctly illustrated that relative to the chromatographic determination, smaller constructs were underrepresented or not detected, while larger constructs were highly overrepresented by ELISA. Thus, in the performed ELISA, strong discrimination based on construct sizes was observed.

As shown in FIG. 4, the LC-based determination reached recovery values between 85% and 98% while using the individual nucleoside responses as reference. Although ELISA assays could detect dsRNA and set up in a high throughput manner, they were severely influenced by the size of the dsRNA fragments and the nature of the underlying standard. In contrast, the LC method provided more consistent results across different fragment sizes and was not based on any underlying dsRNA standard. Therefore, employing LC as an alternative method to ELISA for dsRNA detection and quantification provided a more nuanced and comprehensive understanding of dsRNA contamination in mRNA products.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method for quantifying double-stranded nucleic acids in a mixture comprising one or more additional nucleic acids or impurities, the method comprising the steps of:
a. adding a first enzyme solution to the mixture, wherein the first enzyme solution digests the one or more additional nucleic acids or impurities;
b. separating the double-stranded nucleic acids from the digested one or more additional nucleic acids or impurities;
c. adding a second enzyme solution to the separated double-stranded nucleic acids, wherein the second enzyme solution digests the double-stranded nucleic acids into nucleosides; and
d. analyzing the nucleosides by a liquid chromatography (LC) process to quantify the double-stranded nucleic acids in the mixture.

2. The method of claim 1, wherein the mixture comprising one or more additional nucleic acids or impurities is prepared using an in vitro transcription (IVT) reaction, optionally wherein the IVT reaction is performed using a T7 polymerase enzyme and optionally one or more oligonucleotide primers selected from the group consisting of SEQ NOs: 1-11.

3.-4. (canceled)

5. The method of claim 2, wherein the additional nucleic acids in the mixture comprise single-stranded nucleic acids,
optionally wherein the single-stranded nucleic acids are linear or circular,
optionally wherein the single-stranded nucleic acids are mRNA,
optionally wherein the mRNA comprises a modified nucleotide, and
optionally wherein the modified nucleotide is 1-methyl-pseudouridine.

6.-9. (canceled)

10. The method of claim 5, wherein the first enzyme solution comprises a recombinant protein fusion of RNase I and maltose-binding protein (RNase I_{f}).

11. The method of claim 10, wherein the mixture is incubated with the first enzyme solution for about 50 minutes to about 300 minutes, optionally wherein the mixture is incubated with the first enzyme solution for about 210 minutes.

12. (canceled)

13. The method of claim 11, wherein the maximal enzyme to substrate ratio is from about 5 units to about 35 units per microgram of substrate, optionally wherein the maximal enzyme to substrate ratio is 20 units per microgram of substrate.

14. (canceled)

15. The method of claim 13, wherein following step b, the separated double-stranded nucleic acids comprise less than about 15% of the one or more additional nucleic acids or impurities.

16. The method of claim 15, wherein the second enzyme solution is added to the separated double-stranded nucleic acids for about 30 minutes to about 240 minutes, optionally wherein the second enzyme solution digests at least 90% of the double-stranded nucleic acids into nucleosides.

17. (canceled)

18. The method of claim 16, wherein the LC process is reverse phase liquid chromatography (RP-LC), optionally wherein the RP-LC process comprises eluting the nucleosides from an RP-LC apparatus with a mobile phase solution comprising a mixture of a first solvent solution and a second solvent solution.

19. (canceled)

20. The method of claim 18, wherein the first solvent solution comprises one or more solvents selected from the group consisting of: acetonitrile; ammonium acetate; ethanol; formic acid; hexylene glycol; isopropanol; methanol; tetrahydrofuran; and water, optionally wherein the first solvent solution comprises ammonium acetate and formic acid, optionally wherein the first solvent solution comprises 20 mM ammonium acetate and 0.1% formic acid.

21.-22. (canceled)

23. The method of claim 18, wherein the second solvent solution comprises one or more solvents selected from the group consisting of: acetonitrile; ammonium acetate; ethanol; formic acid; hexylene glycol; isopropanol; methanol; tetrahydrofuran; and water, optionally wherein the second solvent solution comprises methanol.

24. (canceled)

25. The method of claim 23, wherein the second solvent solution in the mobile phase solution mixture increases in concentration from about 1% to about 100% during the RP-LC process,
optionally wherein the concentration of the second solvent solution in the mobile phase solution mixture:
a) increases from about 2% to about 21% over about 6.5 minutes,
b) increases from about 21% to about 100% over about 0.45 minutes, and
c) decreases from about 100% to about 0% immediately thereafter.

26. (canceled)

27. The method of claim 23, wherein step d further comprises comparing the double-stranded nucleic acids analyzed by the RP-LC process with reference nucleosides to quantify the double-stranded nucleic acids,
optionally wherein step b further comprises an additional step of characterizing a sample of the separated double-stranded nucleic acids by an ion pair reverse phase liquid chromatography (IP-RP-LC) process, and
optionally wherein the IP-RP-LC process comprises eluting the nucleosides from an IP-RP-LC apparatus with a mobile phase solution comprising a mixture of a first solvent solution and a second solvent solution.

28.-29. (canceled)

30. The method of claim 27, wherein the first solvent solution comprises:
one or more solvents selected from the group consisting of:
acetonitrile; ammonium acetate; ethanol; formic acid; hexylene glycol; isopropanol; methanol; tetrahydrofuran; and water; or
one or more ion pairs selected from the group consisting of:
hexylammonium acetate; triethylammonium acetate; tetrabutylammonium phosphate; and dibutylammonium acetate,
optionally wherein the concentration of each of the one or more ion pairs in the first solvent solution is from about 1 mM to about 100 mM,
optionally wherein the first solvent solution comprises 25 mM hexylammonium acetate.

31.-32. (canceled)

33. The method of claim 30, wherein the second solvent solution comprises one or more solvents selected from the group consisting of: acetonitrile; ammonium acetate; ethanol; formic acid; hexylene glycol; isopropanol; methanol; tetrahydrofuran; and water, optionally wherein the second solvent solution comprises acetonitrile.

34. (canceled)

35. The method of claim 33, wherein the second solvent solution in the mobile phase solution mixture increases in concentration from about 1% to about 100% during the IP-RP-LC process,
optionally wherein the concentration of the second solvent solution in the mobile phase solution mixture:
a) remains at about 5% over about 2.5 minutes;
b) increases from about 5% to about 50% over about 12.5 minutes;
c) increases from about 40% to about 45% over about 5 minutes;
d) increases from about 45% to about 80% over about 5 minutes;
e) remains at about 80% for about 5 minutes, and
f) decreases from about 80% to about 5% immediately thereafter.

36. (canceled)

37. The method of claim 35, wherein the double-stranded nucleic acid is linear or circular, optionally wherein the double-stranded nucleic acid is dsRNA, optionally wherein the double-stranded nucleic acid is dsDNA.

38.-39. (canceled)

40. The method of claim 37, wherein the double-stranded nucleic acids are about 20 bp to about 5000 bp, as characterized by IP-RP-LC analysis.

41. The method of claim 1, further comprising one or more steps of determining the nucleic acid sequences of the separated double-stranded nucleic acids.


SEQUENCE LISTING (11 sequences)

SEQ ID NO: 1 - DNA, 29 bp, synthetic construct
  source 1..29: mol_type = other DNA
taatacgactcactatagggagacccaag

SEQ ID NO: 2 - DNA, 24 bp, synthetic construct
  source 1..24: mol_type = other DNA
ctgtacttcttgtccatggtggcg

SEQ ID NO: 3 - DNA, 47 bp, synthetic construct
  source 1..47: mol_type = other DNA
taatacgactcactatagggagacgccaccatggacaagaagtacag

SEQ ID NO: 4 - DNA, 26 bp, synthetic construct
  source 1..26: mol_type = other DNA
ctgtccaccagtttctttctcaggtg

SEQ ID NO: 5 - DNA, 49 bp, synthetic construct
  source 1..49: mol_type = other DNA
taatacgactcactatagggagactgtccaccagtttctttctcaggtg

SEQ ID NO: 6 - DNA, 27 bp, synthetic construct
  source 1..27: mol_type = other DNA
gtatctcttgatcatagaggcgctcag

SEQ ID NO: 7 - DNA, 48 bp, synthetic construct
  source 1..48: mol_type = other DNA
taatacgactcactatagggagagtatctcttgatcatagaggcgctc

SEQ ID NO: 8 - DNA, 20 bp, synthetic construct
  source 1..20: mol_type = other DNA
gtgtatctccgccgcttcag

SEQ ID NO: 9 - DNA, 38 bp, synthetic construct
  source 1..38: mol_type = other DNA
taatacgactcactatagggagagtgtatctccgccgc

SEQ ID NO: 10 - DNA, 20 bp, synthetic construct
  source 1..20: mol_type = other DNA
ccagctgagacaggtcgatc

SEQ ID NO: 11 - DNA, 40 bp, synthetic construct
  source 1..40: mol_type = other DNA
taatacgactcactatagggagaccagctgagacaggtcg